(19) United States
(12) Patent Application Publication
(10) Pub. No.: US 2024/0366586 A1
(43) Pub. Date: Nov. 7, 2024

(54) METHODS AND AGENTS FOR MODULATING THE IMMUNE RESPONSE

(71) Applicant: The Regents of the University of California, Oakland, CA (US)

(72) Inventors: Stephen T. SMALE, Los Angeles, CA (US); Philip O. SCUMPIA, Culver City, CA (US); Robert D. DAMOISEAUX, Los Angeles, CA (US); Kevin DOTY, Los Angeles, CA (US)

(73) Assignee: The Regents of the University of California, Oakland, CA (US)

(21) Appl. No.: 18/687,257

(22) PCT Filed: Aug. 31, 2022
(86) PCT No.: PCT/US22/42200

§ 371 Date: Feb. 27, 2024

Related U.S. Application Data
(60) Provisional application No. 63/239,048, filed on Aug. 31, 2021.

(51) Int. Cl. A61K 31/4709; A61K 31/121; A61K 31/345; A61K 31/357; A61K 31/381; A61K 31/439; A61K 31/4704; A61K 31/4741; A61K 45/06; A61P 17/06; A61P 17/14

(52) U.S. Cl. CPC A61K 31/4709; A61K 31/121; A61K 31/345; A61K 31/357; A61K 31/381; A61K 31/439; A61K 31/4704; A61K 31/4741; A61K 45/06; A61P 17/06; A61P 17/14

(57) ABSTRACT

Described are agents and methods for modulating an immune response. More particularly, described are various agents and methods for selectively regulating expression of the gene Il12b in live animals, including human patients. Aberrant expression of Il12b is associated with autoinflammatory diseases including psoriasis, chronic eczema, vitiligo, lichen planus, systemic lupus erythematosus, Behçet's disease, and alopecia. Selective modulation of Il12b offers a new avenue for treatment of such diseases.

### CROSS-REFERENCE TO RELATED APPLICATIONS

This application claims priority to U.S. Provisional Patent Application Ser. No. 63/239,048, filed Aug. 31, 2021, which is incorporated by reference herein in its entirety.

### GOVERNMENT INTEREST STATEMENT

This disclosure was made with government support under Grant Number AI076921 and AR066545, awarded by the National Institutes of Health. The government has certain rights in the disclosure.

### FIELD OF INTEREST

The present application relates generally to methods and agents for modulating an immune response, especially an inflammatory response in a mammal.

### BACKGROUND

Interleukins (ILs) are a diverse group of secreted cytokines traditionally associated with vertebrate white blood cell immune response. The human immune system depends in great part on ILs and their activities. Relatedly, a great number of disorders and pathologies are associated with deficiency, surplus, and dysfunction of one or more ILs.

IL-12p40 is a protein subunit that is a shared component of both IL-12 and IL-23. The majority of ILs are synthesized by CD4⁺ “helper” T cells (T_{H} cells), and these ILs have been shown to be critical for generating T_{H}1 and T_{H}17 adaptive immune responses, respectively. See A. Berger, Th1 and Th2 responses: what are they?, BRIT. MED. J. 2000 Aug. 12; 321(7258): 424; L. A. Tesmer et al., Th17 cells in human disease, IMMUNOL. REV. 2008 June; 223: 87-113.

Aberrant expression of IL-12p40 impacts several autoinflammatory diseases, including, e.g., psoriasis, and thus IL-12p40 is an important pharmacologic target for treatment of such diseases. Accordingly, investigators have sought regulators of the gene that encodes IL-12p40, Il12b.

One problem with many traditional experiments investigating gene-modulation in systems such as Il12b is that investigators cannot discern between different mechanisms affecting gene expression. Eukaryotic nuclear DNA is coiled up in histones forming a coiled-DNA-histone complex called “chromatin”; much recent discovery in the field of epigenetics has found that gene expression very often depends on whether a relevant gene is located in a tightly coiled “closed” chromatin or a loosely coiled “open” chromatin. Similarly, some modulators of gene expression can access a gene target on both open and closed histones, while other modulators can only access a gene target on open histones. Further, some modulators of gene expression operate by modifying histones, meaning that the modulator may be effective on a histone-bound version of the gene, but would be useless on a version of the gene unbound to a histone.

Consistent with such experimental shortcomings, existing Il12b-based treatments have significant off-target effects. Thus, there is a significant unmet need for more selective Il12b regulators. A novel approach for identifying improved Il12b regulators is the use of a dual fluorescence reporter system that allows investigators to discern between open-chromatin modulators of Il12b and closed-chromatin modulators of Il12b. Such an approach would identify new compounds and methods for modulating Il12b and treating autoinflammatory disease, with a greater understanding of the mechanism of action, which will afford doctors and patients more, and better, treatment options.

### BRIEF SUMMARY

The present description provides methods and agents for treating an inflammatory disease in a live animal such as a human. In an aspect, the present description provides a method of treating an inflammatory disease in a live animal, the method of treatment comprising administering a therapeutically effective dose of a selective Il12b inhibitor, wherein the selective Il12b inhibitor may be a compound selected from the Formulas (I)-(IV) below.

In one embodiment, a compound is provided having Formula (I) or a pharmaceutical composition is provided comprising a compound of Formula (I):

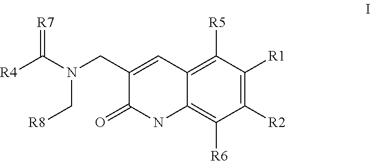

wherein R1 and R2 are independently hydrogen, alkyl, or alkyloxy; or R1 and R2 taken together is methylenedioxy or ethylenedioxy;
R8 is thienyl, furyl or 4-halophenyl;
R4 is 4-alkylphenyl, 2,4-dialkoxyphenyl, 4-alkylphenylamino, 4-alkoxyphenylamino, benzylamino, furylalkylamino or thienyl;
R5 and R6 are independently hydrogen or alkyl;
and R7 is sulfur or oxygen.

In some embodiments, R1 and R2 are independently hydrogen, ethoxy, methoxy or methyl. In some embodiments, R1 and R2 taken together is ethylenedioxy. In some embodiments, R8 is 2-thienyl, 2-furyl or 4-fluorophenyl. In some embodiments, R4 is 4-methylphenyl, 4-t-butyl-phenyl, 2,4-dimethoxyphenyl, 4-methylphenylamino, 4-ethoxyphenylamino, benzylamino, furylmethylamino or thienyl. In some embodiments, furylmethylamino is 2-furylmethylamino. In some embodiments, R5 and R6 are independently hydrogen or methyl. In some embodiments, R7 is sulfur or oxygen.

Non-limiting examples of compounds of Formula (I) include N-(2-furylmethyl)-N-[(6-methoxy-2-oxo-1H-quinolin-3-yl)methyl]-4-methyl-benzamide; N-[(5,8-dimethyl-2-oxo-1H-quinolin-3-yl)methyl]-N-[(4-fluorophenyl)methyl]-3,5-dimethoxybenzamide; 4-tert-butyl-N-[(6-ethoxy-2-oxo-1H-quinolin-3-yl)methyl]-N-(furan-2-ylmethyl)benzamide; 1-(furan-2-ylmethyl)-3-(4-methylphenyl)-1-[(7-oxo-3,6-dihydro-2H-[1,4]dioxino[2,3-g]quinolin-8-yl)methyl]urea; 1-[(6-ethoxy-2-oxo-1H-quinolin-3-yl)methyl]-1-(furan-2-ylmethyl)-3-(4-methoxyphenyl)urea; 3-(4-ethoxyphenyl)-1-(furan-2-ylmethyl)-1-[(7-methyl-2-oxo-1H-quinolin-3-yl)methyl]thiourea; 1-(furan-2-ylmethyl)-3-(4-methylphenyl)-1-[(6-oxo-5H-[1,3]dioxolo[4,5-g]quinolin-7-yl)methyl]urea; 3-benzyl-1-(furan-2-ylmethyl)-1-[(6-oxo-5H-[1,3]dioxolo[4,5-g]quinolin-7-yl)methyl]urea; 3-benzyl-1-[(6-ethyl-2-oxo-1H-quinolin-3-yl)methyl]-1-(furan-2-ylmethyl)thiourea; 1-[(5,8-dimethyl-2-oxo-1H-quinolin-3-yl)methyl]-1,3-bis(furan-2-ylmethyl)thiourea; and N-[(6-ethoxy-2-oxo-1H-quinolin-3-yl)methyl]-N-(furan-2-ylmethyl)thiophene-2-carboxamide.

In some embodiments, a compound is provided having Formula (II) or a pharmaceutical composition is provided comprising a compound of Formula (II):

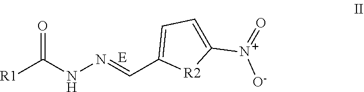

wherein R1 is aryl optionally substituted by one or more halo, alkyl or arylalkyl; and
R2 is oxygen or sulfur.

In some embodiments, R1 is phenyl optionally substituted with one or more halo or alkyl. In some embodiments, R1 is 4-halophenyl. In some embodiments, R1 is 3-halophenyl. In some embodiments, R1 is 4-(1,1′-biphenyl). In some embodiments, R1 is naphthyl. In some embodiments, R1 is 1-naphthyl. In some embodiments, R1 is phenylalkyl. In some embodiments, R1 is phenylethyl. In some embodiments, R1 is naphthylalkyl. In some embodiments, R1 is 1-naphthylmethyl.

Non-limiting example of compounds of Formula (II) include 3-fluoro-N′-[(5-nitro-2-thienyl)methylene]benzohydrazide; N—[(Z)-(5-nitrothiophen-2-yl)methylideneamino]-3-phenylpropanamide; II-3: N—[(Z)-(5-nitrofuran-2-yl)methylideneamino]-4-phenylbenzamide; II-4: 3-chloro-N-[(E)-1-(5-nitrofuran-2-yl)ethylideneamino]benzamide; 2-(4-chlorophenyl)-N—[(Z)-(5-nitrofuran-2-yl)methylideneamino]acetamide; and N—[(Z)-(5-nitrofuran-2-yl)methylideneamino]naphthalene-1-carboxamide.

In some embodiment, a compound of Formula (III) is provided or a pharmaceutical composition comprising a compound of Formula (III) is provided:

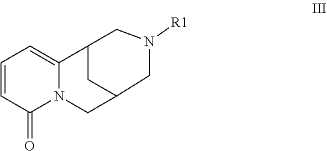

wherein R1 is benzyl optionally substituted with one or more halo, alkoxy, hydroxy, nitro, alkylthio, ethylenedioxy, aryl or benzyloxy groups, or R1 is a 1,3a-dihydropyrenylmethyl.

In some embodiments, R1 is benzyl substituted with one or more halo, alkoxy or hydroxy groups. In some embodiments, R1 is substituted with a halo and an alkoxy group. In some embodiments, the compound of Formula (III) has a 1R,9S configuration.

Non-limiting example of compounds of Formula (III) include 11-(5-hydroxy-2-nitrobenzyl)-7,11-diazatricyclo[7.3.1.0˜2,7˜]trideca-2,4-dien-6-one and (1R,9S)-11-(pyren-1-ylmethyl)-7,11-diazatricyclo[7.3.1.0^{2,7}]trideca-2,4-dien-6-one.

In some embodiments, a compound is provided of Formula (IV) or a pharmaceutical composition comprising a compound of Formula (IV) is provided:

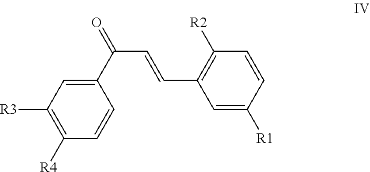

wherein R1 is halo or alkyloxy;
R2 and R3 are independently alkyloxy; and
R4 is hydrogen, alkyl or alkoxy.

In some embodiments, R1 is bromo or methoxy. In some embodiments, R2 is methoxy or ethoxy. In some embodiments, R3 is methoxy. In some embodiments, R4 is methyl or methoxy.

Non-limiting example of compounds of Formula (IV) include 3-(5-bromo-2-methoxyphenyl)-1-(3,4-dimethoxyphenyl)-2-propen-1-one; (E)-3-(5-bromo-2-methoxyphenyl)-1-(2,3-dihydro-1,4-benzodioxin-6-yl)prop-2-en-1-one; (E)-3-(2,5-dimethoxyphenyl)-1-(4-methylphenyl)prop-2-en-1-one; (E)-3-(3-chlorophenyl)-1-(3,4-dimethoxyphenyl)prop-2-en-1-one; and (E)-3-(2,3-dimethoxyphenyl)-1-(4-ethoxyphenyl)prop-2-en-1-one.

In an aspect, the present description provides a method of treating an inflammatory disease in a live animal such as a human, the method of treatment comprising administering a therapeutically effective dose of a selective Il12b inhibitor, wherein the selective Il12b inhibitor may be selected from compounds consisting of: 1-(2-nitro-1-propen-1-yl)naphthalene; 2-{[(5-nitro-2-thienyl)methylene]amino}benzamide; 1-ethoxy-4-(2-nitrovinyl)benzene; 3-(5-bromo-2-methoxyphenyl)-1-(3,4-dimethoxyphenyl)-2-propen-1-one; 2-(1H-benzimidazol-2-yl)-1-phenyl-3-(3-pyridinyl)-2-propen-1-one; 5,6-dimethyl-N-[(5-nitro-2-furyl)methylene]-1H-benzimidazol-1-amine; 11-(5-hydroxy-2-nitrobenzyl)-7,11-diazatricyclo[7.3.1.0˜2,7˜]trideca-2,4-dien-6-one; 6-chloro-2-trichloromethyl)-4H-chromen-4-one; N-(5-bromo-2-biphenylyl)acetamide; diethyl [(1,3-dioxo-2,3-dihydro-1H-inden-5-yl)methylene]malonate; 5-ethylphenazin-5-ium ethyl sulfate; and 3-fluoro-N′-[(5-nitro-2-thienyl)methylene]benzohydrazide, or an analogue of any of the foregoing.

In an aspect, the present description provides a method of treating an inflammatory disease in a live animal such as a human, the method of treatment comprising administering a therapeutically effective dose of a selective Il12b inhibitor, wherein the selective Il12b inhibitor may be selected from 1-(2-nitro-1-propen-1-yl)naphthalene; 2-{[(5-nitro-2-thienyl)methylene]amino}benzamide; 1-ethoxy-4-(2-nitrovinyl)benzene; 3-(5-bromo-2-methoxyphenyl)-1-(3,4-dimethoxyphenyl)-2-propen-1-one; 2-(1H-benzimidazol-2-yl)-1-phenyl-3-(3-pyridinyl)-2-propen-1-one; 5,6-dimethyl-N-[(5-nitro-2-furyl)methylene]-1H-benzimidazol-1-amine; 11-(5-hydroxy-2-nitrobenzyl)-7,11-diazatricyclo[7.3.1.0˜2,7˜]trideca-2,4-dien-6-one; 6-chloro-2-(trichloromethyl)-4H-chromen-4-one; N-(5-bromo-2-biphenylyl)acetamide; diethyl [(1,3-dioxo-2,3-dihydro-1H-inden-5-yl)methylene]malonate; 5-ethylphenazin-5-ium ethyl sulfate; 1,2-dimethoxy-4-[(E)-2-nitroethenyl]benzene; 4-{[(1E,2E)-3-(5-nitrofuran-2-yl)prop-2-en-1-ylidene]amino}-5-phenyl-4H-1,2,4-triazole-3-thiol; (E)-1-ethoxy-2-(2-nitroprop-1-en-1-yl)benzene; (5E)-1-(3,4-Dimethylphenyl)-5-{[1-(4-methoxyphenyl)-1H-pyrrol-2-yl]methylidene}-2-thioxodihydropyrimidine-4,6(1H,5H)-dione; (1E,4E)-1,5-Bis(2-methoxyphenyl)penta-1,4-dien-3-one; 3-Fluoro-N′-[(E)-(5-nitrothiophen-2-yl)methylidene]benzohydrazide; 3-Methyl-5-(6-nitro-benzo[1,3]dioxol-5-ylmethylene)-2-thioxo-thiazolidin-4-one; 1-(2,3-Dihydro-benzo[1,4]dioxin-6-yl)-3-(3,4,5-trimethoxy-phenyl)-propenone; N-(2-Furylmethyl)-N-[(6-methoxy-2-oxo-1H-quinolin-3-yl)methyl]-4-methyl-benzamide; and N-[2-Chloro-5-(trifluoromethyl)phenyl]-5-nitrofuran-2-carboxamide, or an analogue of any of the foregoing.

In any embodiment, the selective Il12b inhibitor may comprise any combination of a compound of any Formulas (I)-(IV) or any compound described herein.

The present description provides methods and agents for treating an inflammatory disease in a live animal such as a human. In an aspect, the present description provides a method of treating an inflammatory disease in a live animal such as a human, the method of treatment comprising administering a therapeutically effective dose of a selective Il12b inhibitor, wherein the selective Il12b inhibitor may be selected from: 1-(2-nitro-1-propen-1-yl)naphthalene; 2-{[(5-nitro-2-thienyl)methylene]amino}benzamide; 1-ethoxy-4-(2-nitrovinyl)benzene; 3-(5-bromo-2-methoxyphenyl)-1-(3,4-dimethoxyphenyl)-2-propen-1-one; 2-(1H-benzimidazol-2-yl)-1-phenyl-3-(3-pyridinyl)-2-propen-1-one; 5,6-dimethyl-N-[(5-nitro-2-furyl)methylene]-1H-benzimidazol-1-amine; 11-(5-hydroxy-2-nitrobenzyl)-7,11-diazatricyclo[7.3.1.0˜2,7˜]trideca-2,4-dien-6-one; 6-chloro-2-trichloromethyl)-4H-chromen-4-one; N-(5-bromo-2-biphenylyl)acetamide; diethyl [(1,3-dioxo-2,3-dihydro-1H-inden-5-yl)methylene]malonate; 5-ethylphenazin-5-ium ethyl sulfate; and 3-fluoro-N′-[(5-nitro-2-thienyl)methylene]benzohydrazide. In any embodiment, the selective Il12b inhibitor may comprise any combination of the above-listed compounds.

In another aspect, the present description provides a method of treating an inflammatory disease in a live animal such as a human, the method comprising administering a therapeutically effective dose of a selective Il12b inhibitor, wherein the selective Il12b inhibitor is a Gα G-protein coupled receptor (GPCR) activator that leads to cyclic adenosine monophosphate (cAMP) production. In some embodiments, the inhibiting occurs in immune cells or in innate immune cells.

In another aspect, the present description provides a method of treatment of an inflammatory disease in a live animal such as a human, the method comprising administering a therapeutically effective dose of a selective Il12b inhibitor, wherein the selective Il12b inhibitor is a Gαs-subtype GPCR activator. In some embodiments, the inhibiting occurs in immune cells or in innate immune cells.

In another aspect, the present description provides a method of treatment of an inflammatory disease in a live animal such as a human, the method comprising administering a therapeutically effective dose of a selective Il12b inhibitor, wherein the selective Il12b inhibitor is a Gβi/o-subtype GPCR activator. In some embodiments, the inhibiting occurs in immune cells or in innate immune cells.

In another aspect, the present description provides a method of treatment of an inflammatory disease in a live animal such as a human, the method comprising administering a therapeutically effective dose of a selective Il12b inhibitor, wherein the selective Il12b inhibitor is a Gαq/11-subtype GPCR activator. In some embodiments, the inhibiting occurs in immune cells or in innate immune cells.

In another aspect, the present description provides a method of treatment of an inflammatory disease in a live animal such as a human, the method comprising administering a therapeutically effective dose of a selective Il12b inhibitor, wherein the selective Il12b inhibitor is a Gαl2/13-subtype GPCR activator. In some embodiments, the inhibiting occurs in immune cells or in innate immune cells.

In yet another aspect, the present description provides a method of treating an inflammatory disease in a live animal such as a human, the method comprising administering a therapeutically effective dose of a selective Il12b inhibitor, wherein the selective Il12b inhibitor is a β adrenergic activator. In any embodiment, the β adrenergic activator may be a β adrenergic agonist. In such embodiments, the β adrenergic agonist may be selected from the group consisting of: denopamine, dobutamine, dopexamine, epinephrine, isoprenaline, isoproterenol, prenalterol, xamoterol, arformoterol, buphenine, clenbuterol, fenoterol, formoterol, isoetarine, levosalbutamol, levalbuterol, orciprenaline, metaproterenol, pirbuterol, procaterol, ritodrine, salbutamol, albuterol, salmeterol, and terbutaline. In any embodiment, the β adrenergic agonist may comprise any combination of the above-listed compounds. In some embodiments, the inhibiting occurs in immune cells or in innate immune cells.

In still another aspect the present description provides a method of treating an inflammatory disease in a live animal such as a human, the method comprising administering a therapeutically effective dose of a selective Il12b inhibitor, wherein the selective Il12b inhibitor is a β₂ adrenergic activator. In any embodiment of the method of treatment, the 32 adrenergic agonist may be selected from the list of compounds consisting of: arformoterol, buphenine, clenbuterol, dopexamine, epinephrine, fenoterol, formoterol, isoetarine, isoprenaline, isoproterenol, levosalbutamol, levalbuterol, orciprenaline, metaproterenol, pirbuterol, procaterol, ritodrine, salbutamol, albuterol, salmeterol, and terbutaline. In any embodiment, the β₂ adrenergic agonist may comprise any combination of the above-listed compounds. In some embodiments, the inhibiting occurs in immune cells or in innate immune cells.

In some embodiments, the present description provides a method of treating an inflammatory disease in a live animal such as a human, the method of treatment comprising administering a therapeutically effective dose of two or more selective Il12b inhibitors, wherein the two or more selective Il12b inhibitor may be selected from the list of compounds consisting of:
a compound of any one or more of Formulas (I)-(IV); 1-(2-nitro-1-propen-1-yl)naphthalene; 2-{[(5-nitro-2-thienyl)methylene]amino}benzamide; 1-ethoxy-4-(2-nitrovinyl)benzene; 3-(5-bromo-2-methoxyphenyl)-1-(3,4-dimethoxyphenyl)-2-propen-1-one; 2-(1H-benzimidazol-2-yl)-1-phenyl-3-(3-pyridinyl)-2-propen-1-one; 5,6-dimethyl-N-[(5-nitro-2-furyl)methylene]-1H-benzimidazol-1-amine; 11-(5-hydroxy-2-nitrobenzyl)-7,11-diazatricyclo[7.3.1.0˜2,7˜]trideca-2,4-dien-6-one; 6-chloro-2-(trichloromethyl)-4H-chromen-4-one; N-(5-bromo-2-biphenylyl)acetamide; diethyl [(1,3-dioxo-2,3-dihydro-1H-inden-5-yl)methylene]malonate; 5-ethylphenazin-5-ium ethyl sulfate; 1,2-dimethoxy-4-[(E)-2-nitroethenyl]benzene; 4-{[(1E,2E)-3-(5-nitrofuran-2-yl)prop-2-en-1-ylidene]amino}-5-phenyl-4H-1,2,4-triazole-3-thiol; (E)-1-ethoxy-2-(2-nitroprop-1-en-1-yl)benzene; (5E)-1-(3,4-Dimethylphenyl)-5-{[1-(4-methoxyphenyl)-1H-pyrrol-2-yl]methylidene}-2-thioxodihydropyrimidine-4,6(1H,5H)-dione; (1E,4E)-1,5-Bis(2-methoxyphenyl)penta-1,4-dien-3-one; 3-Fluoro-N′-[(E)-(5-nitrothiophen-2-yl)methylidene]benzohydrazide; 3-Methyl-5-(6-nitro-benzo[1,3]dioxol-5-ylmethylene)-2-thioxo-thiazolidin-4-one; 1-(2,3-Dihydro-benzo[1,4]dioxin-6-yl)-3-(3,4,5-trimethoxy-phenyl)-propenone; N-(2-Furylmethyl)-N-[(6-methoxy-2-oxo-1H-quinolin-3-yl)methyl]-4-methyl-benzamide; and N-[2-Chloro-5-(trifluoromethyl)phenyl]-5-nitrofuran-2-carboxamide, or an analogue of any of the foregoing,
and a β adrenergic agonist. In some embodiments, the β adrenergic agonist is a β₂ adrenergic agonist.

In some embodiments, the present description provides a method of treating an inflammatory disease in a live animal such as a human, the method of treatment comprising administering a therapeutically effective dose of two or more selective Il12b inhibitors, wherein the two or more selective Il12b inhibitor may be selected from the list of compounds consisting of: a compound of any one or more of Formulas (I)-(IV); 1-(2-nitro-1-propen-1-yl)naphthalene; 2-{[(5-nitro-2-thienyl)methylene]amino}benzamide; 1-ethoxy-4-(2-nitrovinyl)benzene; 3-(5-bromo-2-methoxyphenyl)-1-(3,4-dimethoxyphenyl)-2-propen-1-one; 2-(1H-benzimidazol-2-yl)-1-phenyl-3-(3-pyridinyl)-2-propen-1-one; 5,6-dimethyl-N-[(5-nitro-2-furyl)methylene]-1H-benzimidazol-1-amine; 11-(5-hydroxy-2-nitrobenzyl)-7,11-diazatricyclo[7.3.1.0˜2,7˜]trideca-2,4-dien-6-one; 6-chloro-2-(trichloromethyl)-4H-chromen-4-one; N-(5-bromo-2-biphenylyl)acetamide; diethyl [(1,3-dioxo-2,3-dihydro-1H-inden-5-yl)methylene]malonate; 5-ethylphenazin-5-ium ethyl sulfate; 1,2-dimethoxy-4-[(E)-2-nitroethenyl]benzene; 4-{[(E,2E)-3-(5-nitrofuran-2-yl)prop-2-en-1-ylidene]amino}-5-phenyl-4H-1,2,4-triazole-3-thiol; (E)-1-ethoxy-2-(2-nitroprop-1-en-1-yl)benzene; (5E)-1-(3,4-Dimethylphenyl)-5-{[1-(4-methoxyphenyl)-1H-pyrrol-2-yl]methylidene}-2-thioxodihydropyrimidine-4,6(1H,5H)-dione; (1E,4E)-1,5-Bis(2-methoxyphenyl)penta-1,4-dien-3-one; 3-Fluoro-N′-[(E)-(5-nitrothiophen-2-yl)methylidene]benzohydrazide; 3-Methyl-5-(6-nitro-benzo[1,3]dioxol-5-ylmethylene)-2-thioxo-thiazolidin-4-one; 1-(2,3-Dihydro-benzo[1,4]dioxin-6-yl)-3-(3,4,5-trimethoxy-phenyl)-propenone; N-(2-Furylmethyl)-N-[(6-methoxy-2-oxo-1H-quinolin-3-yl)methyl]-4-methyl-benzamide; and N-[2-Chloro-5-(trifluoromethyl)phenyl]-5-nitrofuran-2-carboxamide, or an analogue of any of the foregoing, or denopamine, dobutamine, dopexamine, epinephrine, isoprenaline, isoproterenol, prenalterol, xamoterol, arformoterol, buphenine, clenbuterol, fenoterol, formoterol, isoetarine, levosalbutamol, levalbuterol, orciprenaline, metaproterenol, pirbuterol, procaterol, ritodrine, salbutamol, albuterol, salmeterol, or terbutaline.

In yet another aspect, the present description provides a method of treating an inflammatory disease in a live animal such as a human, comprising administering a therapeutically effective dose of a combination of: (a) a composition comprising a Gα protein coupled receptor activator that activates cyclic AMP signaling; and (b) composition comprising a selective inhibitor of cyclic AMP (cAMP) breakdown. In any embodiment of such method of treatment, the Gα protein coupled receptor activator that activates cyclic AMP signaling may be any of a Gα GPCR activator, Gus-subtype GPCR activator, Gαi/o-subtype GPCR activator, Gαq/11-subtype GPCR activator, Gαl2/13-subtype GPCR activator, β adrenergic activator, β adrenergic agonist, β₂ adrenergic activator or a β₂ adrenergic agonist. In any embodiment, the inhibitor of cyclic AMP breakdown is a phosphodiesterase 4 inhibitor (PDE4 inhibitor). In any embodiment, the PDE4 inhibitor may be a selective PDE4 inhibitor. In any embodiment, the PDE4 inhibitor may be selected from the group consisting of mesembrenone, rolipram, ibudilast, piclimilast, luteolin, drotaverine, roflumilast, crisaborole, apremilast, and any combination thereof.

In any embodiment of the method, each one or both components of a composition may be administered topically in the form of a cream, salve, gel, dermal patch, aqueous solution, or non-aqueous solution.

In yet another aspect, the present description provides a method of treating an inflammatory disease in a live animal comprising administering a therapeutically effective dose of a combination of:
(a) a compound of any one of Formulas (I)-(IV) or a compound selected from among 1-(2-nitro-1-propen-1-yl)naphthalene; 2-{[(5-nitro-2-thienyl)methylene]amino}benzamide; 1-ethoxy-4-(2-nitrovinyl)benzene; 3-(5-bromo-2-methoxyphenyl)-1-(3,4-dimethoxyphenyl)-2-propen-1-one; 2-(1H-benzimidazol-2-yl)-1-phenyl-3-(3-pyridinyl)-2-propen-1-one; 5,6-dimethyl-N-[(5-nitro-2-furyl)methylene]-1H-benzimidazol-1-amine; 11-(5-hydroxy-2-nitrobenzyl)-7,11-diazatricyclo[7.3.1.0˜2,7˜]trideca-2,4-dien-6-one; 6-chloro-2-(trichloromethyl)-4H-chromen-4-one; N-(5-bromo-2-biphenylyl)acetamide; diethyl [(1,3-dioxo-2,3-dihydro-1H-inden-5-yl)methylene]malonate; 5-ethylphenazin-5-ium ethyl sulfate; 1,2-dimethoxy-4-[(E)-2-nitroethenyl]benzene; 4-{[(1E,2E)-3-(5-nitrofuran-2-yl)prop-2-en-1-ylidene]amino}-5-phenyl-4H-1,2,4-triazole-3-thiol; (E)-1-ethoxy-2-(2-nitroprop-1-en-1-yl)benzene; (5E)-1-(3,4-Dimethylphenyl)-5-{[1-(4-methoxyphenyl)-1H-pyrrol-2-yl]methylidene}-2-thioxodihydropyrimidine-4,6(1H,5H)-dione; (1E,4E)-1,5-Bis(2-methoxyphenyl)penta-1,4-dien-3-one; 3-Fluoro-N′-[(E)-(5-nitrothiophen-2-yl)methylidene]benzohydrazide; 3-Methyl-5-(6-nitro-benzo[1,3]dioxol-5-ylmethylene)-2-thioxo-thiazolidin-4-one; 1-(2,3-Dihydro-benzo[1,4]dioxin-6-yl)-3-(3,4,5-trimethoxy-phenyl)-propenone; N-(2-Furylmethyl)-N-[(6-methoxy-2-oxo-1H-quinolin-3-yl)methyl]-4-methyl-benzamide; and N-[2-Chloro-5-(trifluoromethyl)phenyl]-5-nitrofuran-2-carboxamide; and
(b) composition comprising a selective inhibitor of cyclic AMP (cAMP) breakdown.

In any embodiment, the inhibitor of cyclic AMP breakdown is a phosphodiesterase 4 inhibitor (PDE4 inhibitor). In any embodiment, the PDE4 inhibitor may be a selective PDE4 inhibitor. In any embodiment, the PDE4 inhibitor may be selected from the group consisting of mesembrenone, rolipram, ibudilast, piclimilast, luteolin, drotaverine, roflumilast, crisaborole, apremilast, and any combination thereof.

In any embodiment of the method, each one or both components may be administered topically in the form of a cream, salve, gel, dermal patch, aqueous solution, or non-aqueous solution. In some embodiments, the PDE4 inhibitor is administered topically or systemically, such as orally.

In one aspect, a therapeutic combination is provided comprising any of the IL12b inhibitors disclosed herein, and an inhibitor of cAMP breakdown. In some embodiments, the therapeutic composition is a formulation comprising both one or more IL12b inhibitors disclosed herein, and one or more inhibitors of cAMP breakdown. In other embodiments, the IL12b inhibitors disclosed herein, and an inhibitor of cAMP breakdown, are in separate formulations, but administered concurrently or in an overlapping schedule. In some embodiments, the components are administered by the same or different routes of administration. In one embodiment, the IL12b inhibitor of the therapeutic combination is administered topically, and the inhibitor of cAMP breakdown is administered topically or systemically, such as orally. In some embodiments, the therapeutic combination is a synergistic combination, for example, the effectiveness achieved by administration of the combination is greater than the combined effects of the two components administered at the doses or dose regimens in the combination but in the absence of the other agent.

In any of the foregoing embodiments, the inhibiting may occur in immune cells or in innate immune cells. In any of the foregoing embodiments, the selective Il12b inhibitor may act in immune cells or in innate immune cells. In any of the foregoing embodiments, the selective Il12b inhibitor acts in immune cells or in innate immune cells. In some embodiments, the immune cells or innate immune cells are macrophages.

In any aspect or embodiment, the live animal may be a mammal. In any such aspect or embodiment, the mammal may be a human.

In any aspect or embodiment, the inflammatory disease may be any of psoriasis, chronic eczema, vitiligo, lichen planus, cutaneous lupus erythematosus, Behçet's disease, ulcerative colitis, Crohn's disease, or alopecia. In any aspect or embodiment, the inflammatory disease may be an autoimmune disease, such as type 1 diabetes, rheumatoid arthritis, psoriasis and psoriatic arthritis, multiple sclerosis, systemic lupus erythematosus, inflammatory bowel disease including Crohn's disease and ulcerative colitis, Addison's disease, celiac disease, dermatomyositis, Grave's disease, Sjögren's syndrome, Hashimoto thyroiditis, autoimmune vasculitis, multiple sclerosis, myasthenia gravis, and pernicious anemia.

In one embodiment, a compound is provided having Formula (I):

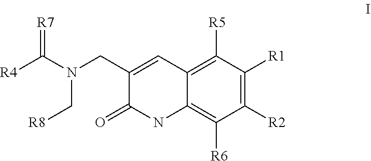

wherein R1 and R2 are independently hydrogen, alkyl, or alkyloxy; or R1 and R2 taken together is methylenedioxy or ethylenedioxy;
R8 is thienyl, furyl or 4-halophenyl;
R4 is 4-alkylphenyl, 2,4-dialkoxyphenyl, 4-alkylphenylamino, 4-alkoxyphenylamino, benzylamino, furylalkylamino or thienyl;
R5 and R6 are independently hydrogen or alkyl;
and R7 is sulfur or oxygen.

In some embodiments, R1 and R2 are independently hydrogen, ethoxy, methoxy or methyl. n some embodiments, R1 and R2 taken together is ethylenedioxy. In some embodiments, R8 is 2-thienyl, 2-furyl or 4-fluorophenyl. In some embodiments, R4 is 4-methylphenyl, 4-t-butyl-phenyl, 2,4-dimethoxyphenyl, 4-methylphenylamino, 4-ethoxyphenylamino, benzylamino, furylmethylamino or thienyl. In some embodiments, furylmethylamino is 2-furylmethylamino. In some embodiments, R5 and R6 are independently hydrogen or methyl. In some embodiments, R7 is sulfur or oxygen.

In some embodiments, a compound is provided having Formula (II):

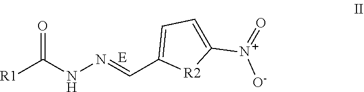

wherein R1 is aryl optionally substituted by one or more halo, alkyl or arylalkyl; and
R2 is oxygen or sulfur.

In some embodiments, R1 is phenyl optionally substituted with one or more halo or alkyl. In some embodiments, R1 is 4-halophenyl. In some embodiments, R1 is 3-halophenyl. In some embodiments, R1 is 4-(1,1′-biphenyl). In some embodiments, R1 is naphthyl. In some embodiments, R1 is 1-naphthyl. In some embodiments, R1 is phenylalkyl. In some embodiments, R1 is phenylethyl. In some embodiments, R1 is naphthylalkyl. In some embodiments, R1 is 1-naphthylmethyl.

In some embodiments, a compound is provided of Formula (III):

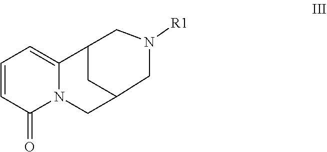

wherein R1 is benzyl optionally substituted with one or more halo, alkoxy, hydroxy, nitro, alkylthio, ethylenedioxy, aryl or benzyloxy groups, or R1 is a 1,3a-dihydropyrenylmethyl.

In some embodiments, R1 is benzyl substituted with one or more halo, alkoxy or hydroxy groups. In some embodiments, R1 is substituted with a halo and an alkoxy group. In some embodiments, the compound of Formula (III) has a 1R,9S configuration.

In some embodiments, a compound of Formula (IV) is provided:

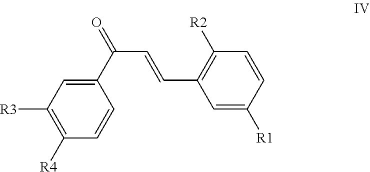

wherein R1 is halo or alkyloxy;
R2 and R3 are independently alkyloxy; and
R4 is hydrogen, alkyl or alkoxy.

In some embodiments, R1 is bromo or methoxy. In some embodiments, R2 is methoxy or ethoxy. In some embodiments, R3 is methoxy. In some embodiment, R4 is methyl or methoxy.

In another embodiment, a pharmaceutical composition is provided of a compound of any one of Formulas (I) through (IV).

### BRIEF SUMMARY OF THE FIGURES

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 depicts a schematic cartoon of a dual-fluorescence reporter macrophage cell line having both a chromatin-bound copy of Il12b fused to a fluorescent reporter and a “naked” copy of Il12b fused to a different fluorescent reporter.

FIG. 2 depicts a block diagram of the investigational approach which discovered key features of the instant agents and methods.

FIG. 3 depicts a diagram of an investigational approach which discovered key features of the instant agents and methods.

FIG. 4A depicts a mapping of a high-throughput screen hits based on eGFP fluorescence (chromatin-expressed Il12b) and DsRed fluorescence (plasmid-expressed Il12b). The red upper-left rectangle highlights test compounds that were selective for inhibiting chromatinized Il12b.

FIG. 4B depicts a mapping of a high-throughput screen based on eGFP fluorescence (chromatin-expressed Il12b) and DsRed fluorescence (plasmid-expressed Il12b) wherein read-outs for certain specific compounds are depicted according to the key. For purposes of interpreting the key in FIG. 4B, UK14 means 1-ethoxy-2-(2-nitro-1-propen-1-yl)benzene (PubChem CID #874429); UK15 means 1-(3,4-dimethylphenyl)-5-{[1-(4-methoxyphenyl)-1H-pyrrol-2-yl]methylene}-2-thioxodihydro-4,6(1H,5H)-pyrimidinedione (CID #2162031); UK16 means 1,5-bis(2-methoxyphenyl)-1,4-pentadien-3-one (CID #830608); UK17 means 3-fluoro-N′-[(5-nitro-2-thienyl)methylene]benzohydrazide (CID #6895129); and UK20 means N-(2-furylmethyl)-N-[(6-methoxy-2-oxo-1H-quinolin-3-yl)methyl]-4-methyl-benzamide (CID #1459058).

FIG. 5 depicts the relative expression levels of minDsRed and BAC-eGFP in samples treated with UK14, UK15, UK16, UK17, and UK20, where these designations have the same meaning as in FIG. 4B.

FIG. 6 depicts relative expression of minDsRed constructs and BAC-eGFP constructs after treatment with a fourfold serial dilution of UK17 (3-fluoro-N′-[(5-nitro-2-thienyl)methylene]benzohydrazide (CID #6895129)), a novel compound selective for inhibition of chromatinized Il12b.

FIG. 7A depicts the effects of UK17 (3-fluoro-N′-[(5-nitro-2-thienyl)methylene]benzohydrazide (CID #6895129)) on lipopolysaccharide-induced genes (RPKM>4 and FC>10) ranked in descending fold-change order (Max RPKM with UK17/Max RPKM without UK17. Il12b ranks in the top 20 most strongly inhibited lipopolysaccharide-induced genes in samples treated with selective inhibitors of chromatinized Il12b. Black dotted lines indicate the mean of the fold change values for all lipopolysaccharide-induced genes. Blue dotted lines represent one standard deviation above or below the mean, and red dotted lines represent two standard deviations above or below the mean.

FIG. 7B depicts a fold change box plot in samples treated with UK17 (3-fluoro-N′-[(5-nitro-2-thienyl)methylene]benzohydrazide (CID #6895129)), classifying lipopolysaccharide-induced genes by gene-regulation cluster as previously described in Tong A. et al., A Stringent Systems Approach Uncovers Gene-Specific Mechanisms Regulating Inflammation, CELL 165 165-179 (2016). Box plots indicate the distribution of fold change values for each cluster. Red lines are drawn at 1 and −1, indicating a 2-fold up or down-regulation of genes. Il12b is labeled in red.

FIG. 8A depicts the effects of UK4 (3-(5-bromo-2-methoxyphenyl)-1-(3,4-dimethoxyphenyl)-2-propen-1-one (CID #5335375)) on lipopolysaccharide-induced genes (RPKM>4 and FC>10) ranked in descending fold-change order (Max RPKM with UK17/Max RPKM without UK17. Il12b ranks in the top 20 most strongly inhibited lipopolysaccharide-induced genes in samples treated with selective inhibitors of chromatinized Il12b. Black dotted lines indicate the mean of the fold change values for all lipopolysaccharide-induced genes. Blue dotted lines represent one standard deviation above or below the mean, and red dotted lines represent two standard deviations above or below the mean.

FIG. 8B depicts a fold change box plot in samples treated with UK4 (3-(5-bromo-2-methoxyphenyl)-1-(3,4-dimethoxyphenyl)-2-propen-1-one (CID #5335375)), classifying lipopolysaccharide-induced genes by gene-regulation cluster as previously described in Tong et al. (2016). Box plots indicate the distribution of fold change values for each cluster. Red lines are drawn at 1 and −1, indicating a 2-fold up or down-regulation of genes. Il12b is labeled in red.

FIG. 9A depicts the effects of UK6 (5,6-dimethyl-N-[(5-nitro-2-furyl)methylene]-1H-benzimidazol-1-amine (CID #6896342)) on lipopolysaccharide-induced genes (RPKM>4 and FC>10) ranked in descending fold-change order (Max RPKM with UK17/Max RPKM without UK17. Il12b ranks in the top 20 most strongly inhibited lipopolysaccharide-induced genes in samples treated with selective inhibitors of chromatinized Il12b. Black dotted lines indicate the mean of the fold change values for all lipopolysaccharide-induced genes. Blue dotted lines represent one standard deviation above or below the mean, and red dotted lines represent two standard deviations above or below the mean.

FIG. 9B depicts a fold change box plot in samples treated with UK6 (5,6-dimethyl-N-[(5-nitro-2-furyl)methylene]-1H-benzimidazol-1-amine (CID #6896342)), classifying lipopolysaccharide-induced genes by gene-regulation cluster as previously described in Tong et al. (2016). Box plots indicate the distribution of fold change values for each cluster. Red lines are drawn at 1 and −1, indicating a 2-fold up or down-regulation of genes. Il12b is labeled in red.

FIG. 10 depicts a heatmap of percent expression of RPKMs as a percent of the maximum RPKM within the DMSO sample for genes with RPKMs>3 and FC>10 in at least one DMSO timepoint. The heatmap represents that for the averaged samples for each gene between Donor 0 IL34 and Donor 0 MCSF. In the left heatmap, red indicates percent expression equal to or higher than 100%. DMSO and PGE2 samples are indicated as such for timepoints 0 hr, 1 hr, 2 hr, and 6 hr. The heatmap on the right shows represents percent inhibition as the difference between PGE2 samples and the DMSO samples. Here, red represents a positive difference equal to or over 100%, indicating induction, and blue representing a negative difference equal to or less than −100%, indicating inhibition. (n=2).

FIG. 11 depicts log 2 of the fold change of RPKMS after PGE2-treatment and those after DMSO treatment are shown in this scatter plot for genes with RPKMs>3 and FC>10 in at least on DMSO timepoint and this graph represents the 6-hour timepoint. A red line is graphed at x=y, where the RPKMs after PGE2-treatment would equal the RPKMs after DMSO treatment and a point near or on this line would indicate similarity between the two numbers. The plot point representing Il12b is indicated as such. (n=2).

FIG. 12 depicts a volcano plot representing the −log₁₀ of the p-value on the y-axis and the log 2 of the fold change from PGE2-treated samples over DMSO-treated samples at the averaged 6-hour timepoint between Donor 0 IL34 and Donor 0 MCSF. Criteria for genes is set at RPKMs over 3 and fold change over 10. Il12b is indicated as such. (n=2)

FIG. 13 depicts a graph representing the RPKMs for Il12b averaged between the two samples from Donor 0. RPKMs after DMSO treatment are graphed in blue, while RPKMs after PGE2 treatment are graphed in red. Error bars are shown in black. (n=2)

FIG. 14 depicts a graph indicating ELISA data for IL12p40 (the protein encoded by Il12b) at 1-hour, 2-hour, 6-hour, and 24-hour timepoints.

FIG. 15 depicts mouse skin of wild type (WT) mice or mice lacking β₁ and β₂ adrenergic receptors (β₁/β₂ KO) 5 days after topically applying imiquimod to their skin. β₁/β₂ KO mice demonstrate a higher psoriasis activity and severity index (PASI) score than WT mice.

FIGS. 16A-16B depict histology of WT mice or β₁/β₂ KO mice 5 days after topically applying imiquimod to their skin. Histology confirms greater thickening and scale of the skin in $1/$2 KO mice.

FIG. 17A depicts psoriasis area and severity index (PASI) clinical scoring of WT mice versus β₁/β₂ KO mice 5 days after topically applying imiquimod to their skin.

FIG. 17B depicts histological scoring of WT mice versus β₁/β₂ KO mice 5 days after topically applying imiquimod to their skin.

FIG. 18 depicts expression levels of IL12p40 (picograms per milligram of skin) in FVB strain mice versus β₁/β₂ KO mice untreated and treated with β₂ selective adrenergic agonist.

FIG. 19 depicts WT mice given intraperitoneal injection of the β₂ selective adrenergic agonist clenbuterol or vehicle control (saline) daily in mice given topical imiquimod to induce psoriasiform dermatitis.

FIG. 20 depicts the PASI clinical scoring PBS-treated WT mice versus clenbuterol-treated WT mice.

FIG. 21 depicts a schematic cartoon of Il12b regulatory pathway as had been understood prior to the present disclosure.

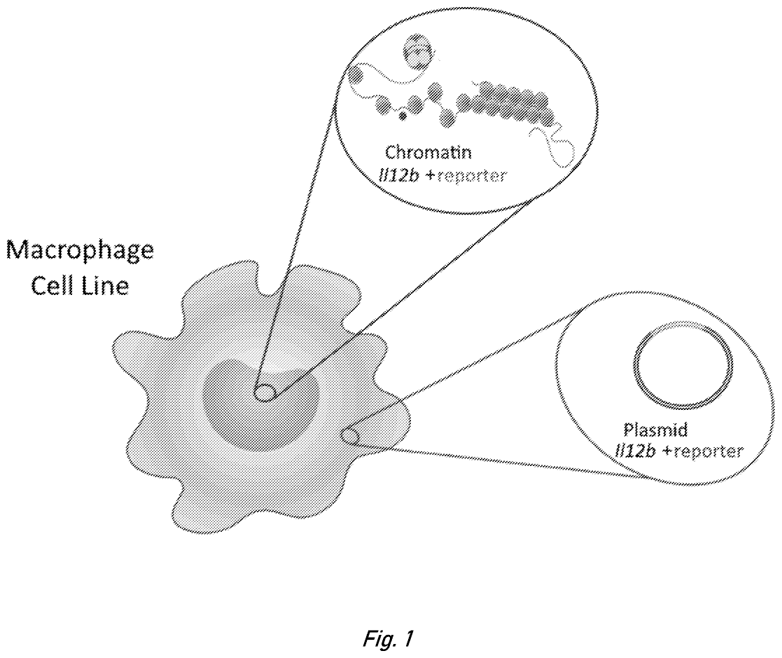

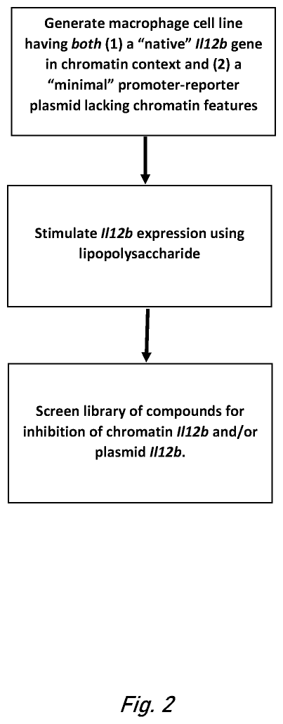

### DETAILED DESCRIPTION

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

As employed above and throughout the disclosure, the following terms and abbreviations, unless otherwise indicated, shall be understood to have the following meanings:

In the present disclosure, the singular forms “a,” “an,” and “the” include the plural reference, and reference to a particular numerical value includes at least that particular value, unless the context clearly indicates otherwise. Thus, for example, a reference to “a compound” is a reference to one or more of such compounds and equivalents thereof known to those skilled in the art, and so forth. The term “plurality”, as used herein, means more than one. When a range of values is expressed, another embodiment includes from the one particular and/or to the other particular value.

Similarly, when values are expressed as approximations, by use of the antecedent “about,” it is understood that the particular value forms another embodiment. All ranges are inclusive and combinable. In the context of the present disclosure, by “about” a certain amount it is meant that the amount is within ±20% of the stated amount, or preferably within ±10% of the stated amount, or more preferably within ±5% of the stated amount.

As used herein, the terms “protein,” “polypeptide,” and “peptide” refer to a molecule comprising amino acids joined via peptide bonds. In general “peptide” is used to refer to a sequence of 20 or less amino acids and “polypeptide” is used to refer to a sequence of greater than 20 amino acids.

As used herein, the term “native” (or wild type) when used in reference to a protein refers to proteins encoded by the genome of a cell, tissue, or organism, other than one manipulated to produce synthetic proteins.

As used herein, the terms “treat”, “treatment”, or “therapy” (as well as different forms thereof) refer to therapeutic treatment, including prophylactic or preventative measures, wherein the object is to prevent or slow down (lessen) an undesired physiological change associated with a disease or condition. Beneficial or desired clinical results include, but are not limited to, alleviation of symptoms, diminishment of the extent of a disease or condition, stabilization of a disease or condition (i.e., where the disease or condition does not worsen), delay or slowing of the progression of a disease or condition, amelioration or palliation of the disease or condition, and remission (whether partial or total) of the disease or condition, whether detectable or undetectable. Those in need of treatment include those already with the disease or condition as well as those prone to having the disease or condition or those in which the disease or condition is to be prevented.

The terms “subject,” “individual,” “live animal,” and “patient” are used interchangeably herein, and refer to an animal, for example a human, to whom treatment with a composition or formulation in accordance with the present disclosure, is provided. The term “subject” as used herein refers to human and non-human animals. The human can be any human of any age. In an embodiment, the human is an adult. In another embodiment, the human is a child. The human can be male, female, pregnant, middle-aged, adolescent, or elderly. The terms include mammals, e.g., humans, non-human primates e.g. a monkey or ape, companion animals (e.g., dogs, cats, rabbits, birds, and the like), farm animals (e.g., cows, sheep, pigs, horses, fowl, and the like) and laboratory animals (e.g., rats, mice, guinea pigs, birds, and the like). In some embodiments, the subject is male human or a female human.

Conditions and disorders in a subject for which a particular drug, compound, composition, formulation (or combination thereof) is said herein to be “indicated” are not restricted to conditions and disorders for which that drug or compound or composition or formulation has been expressly approved by a regulatory authority, but also include other conditions and disorders known or reasonably believed by a physician or other health or nutritional practitioner to be amenable to treatment with that drug or compound or composition or formulation or combination thereof.

Sterile solutions can be prepared by incorporating the compound or molecule, by itself or in combination with other active agents, in the required amount in an appropriate solvent with one or a combination of ingredients enumerated herein, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, one method of preparation is vacuum drying and freeze-drying, which yields a powder of an active ingredient plus any additional desired ingredient from a previously sterile-filtered solution thereof. The preparations for injections are processed, filled into containers such as ampoules, bags, bottles, syringes or vials, and sealed under aseptic conditions according to methods known in the art.

Further, the preparations may be packaged and sold in the form of a kit. Such articles of manufacture will preferably have labels or package inserts indicating that the associated compositions are useful for treating a subject suffering an inflammatory condition or diseases as described herein.

In an aspect, the present description provides a method of treating an inflammatory disease in a live animal, the method of treatment comprising administering a therapeutically effective dose of a selective Il12b inhibitor, wherein the selective Il12b inhibitor may be selected from any one of Formulas (I)-(IV).

In one embodiment, a compound is provided having Formula (I):

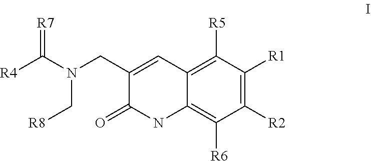

wherein R1 and R2 are independently hydrogen, alkyl, or alkyloxy; or R1 and R2 taken together is methylenedioxy or ethylenedioxy;
R8 is thienyl, furyl or 4-halophenyl;
R4 is 4-alkylphenyl, 2,4-dialkoxyphenyl, 4-alkylphenylamino, 4-alkoxyphenylamino, benzylamino, furylalkylamino or thienyl;
R5 and R6 are independently hydrogen or alkyl;
and R7 is sulfur or oxygen.

In some embodiments, R1 and R2 are independently hydrogen, ethoxy, methoxy or methyl.

In some embodiments, R1 and R2 taken together is ethylenedioxy.

In some embodiments, R8 is 2-thienyl, 2-furyl or 4-fluorophenyl.

In some embodiments, R4 is 4-methylphenyl, 4-t-butyl-phenyl, 2,4-dimethoxyphenyl, 4-methylphenylamino, 4-ethoxyphenylamino, benzylamino, furylmethylamino or thienyl. In some embodiments, furylmethylamino is 2-furylmethylamino.

In some embodiments, R5 and R6 are independently hydrogen or methyl.

In some embodiments, R7 is sulfur or oxygen.

Non-limiting example of compounds of Formula (I) useful for the purposes disclosed herein include:

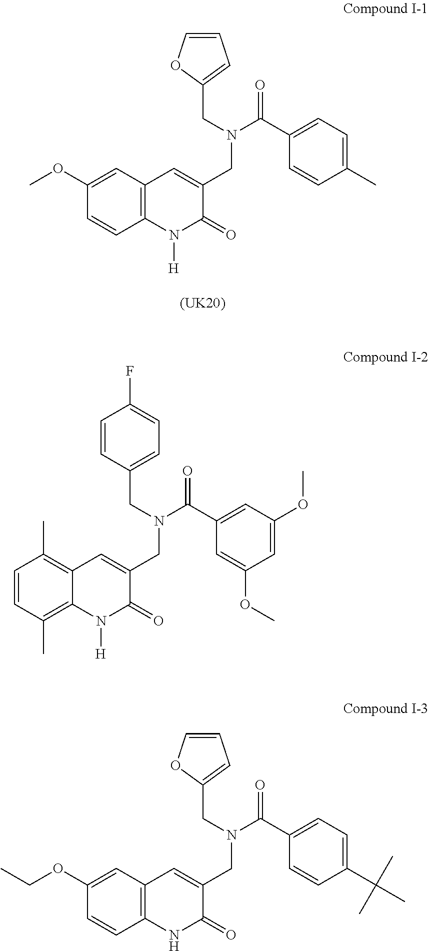

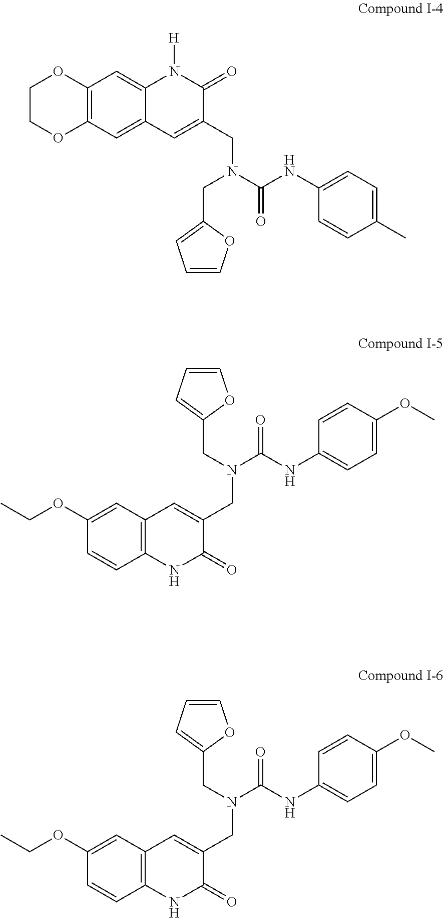

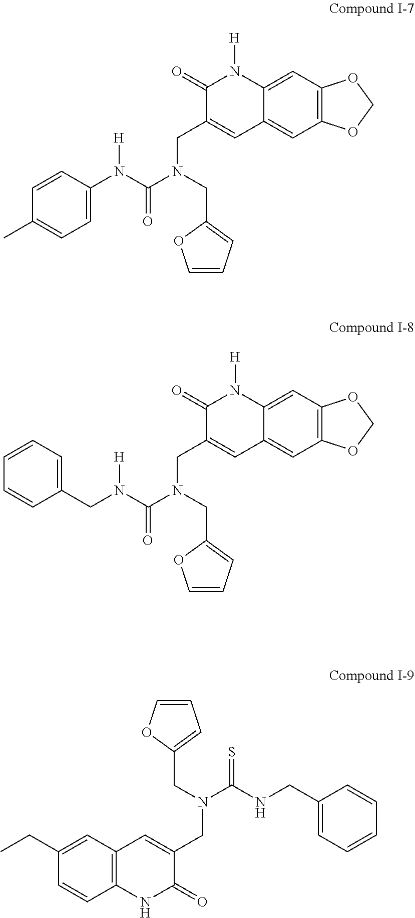 [SMILES: Cc1ccc(NC(=O)N(Cc2ccco2)Cc2cc3cc4c(cc3[nH]c2=O)OCO4)cc1 ; O=C(NCc1ccccc1)N(Cc1ccco1)Cc1cc2cc3c(cc2[nH]c1=O)OCO3 ; CCc1ccc2[nH]c(=O)c(CN(Cc3ccco3)C(=S)NCc3ccccc3)cc2c1]

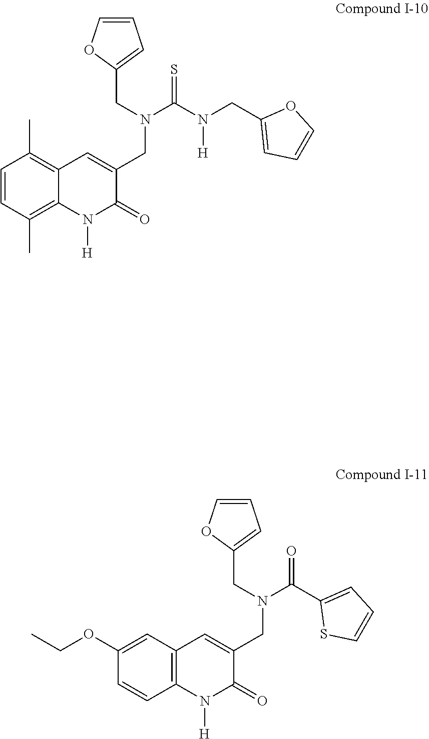 [SMILES: Cc1ccc(C)c2[nH]c(=O)c(CN(Cc3ccco3)C(=S)NCc3ccco3)cc12 ; CCOc1ccc2[nH]c(=O)c(CN(Cc3ccco3)C(=O)c3cccs3)cc2c1]

In some embodiments, the compound of Formula (I) is N-(2-furylmethyl)-N-[(6-methoxy-2-oxo-1H-quinolin-3-yl)methyl]-4-methyl-benzamide.

The disclosure is also directed to a compound of Formula (I) and pharmaceutical compositions comprising a compound of Formula (I). Non-limiting examples include Compounds I-1 through I-11.

Methods for synthesis of compounds of Formula (I) may be found in US-2010035912-A1.

In one embodiment, a compound is provided having Formula (II)

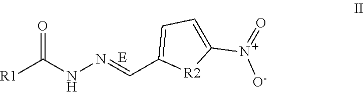

wherein R1 is aryl optionally substituted by one or more halo, alkyl or arylalkyl; and
R2 is oxygen or sulfur.

In some embodiments, R1 is phenyl optionally substituted with one or more halo or alkyl. In some embodiments, R1 is 4-halophenyl. In some embodiments, R1 is 3-halophenyl.

In some embodiments, R1 is 4-(1,1′-biphenyl). In some embodiments, R1 is naphthyl. In some embodiments, R1 is 1-naphthyl.

In some embodiments, R1 is phenylalkyl. In some embodiments, R1 is phenylethyl.

In some embodiments, R1 is naphthylalkyl. In some embodiments, R1 is 1-naphthylmethyl.

Non-limiting example of compounds of Formula (II) useful for the purposes disclosed herein include:

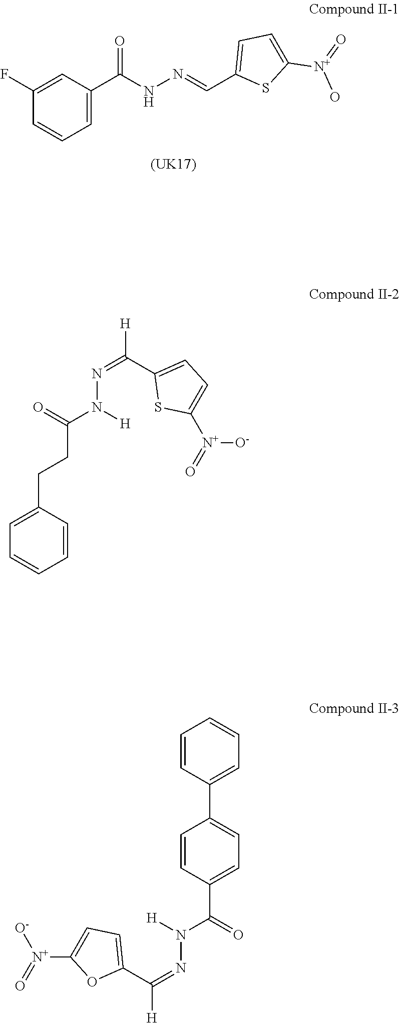

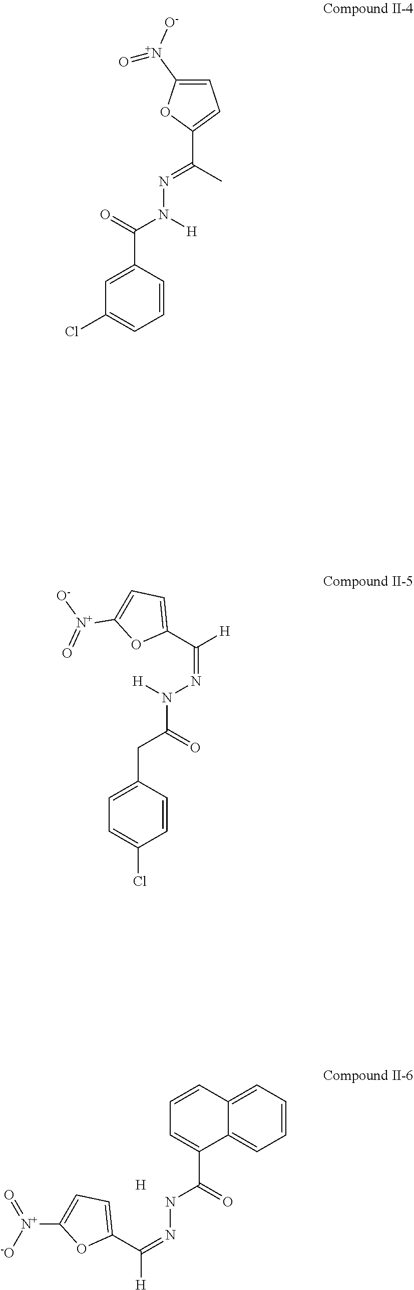

In some embodiments, the compound of Formula (II) is 3-fluoro-N′-[5-nitro-2-thienyl)methylene]benzohydrazide.

Methods for synthesis of compounds of Formula (II) may be found in US-2022009914-A1.

The disclosure is also directed to a compound of Formula (II) and pharmaceutical compositions comprising a compound of Formula (II). Non-limiting examples include Compounds II-1 through II-6.

In some embodiments, a compound is provided having Formula (III):

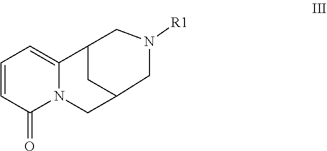

wherein R1 is benzyl optionally substituted with one or more halo, alkoxy, hydroxy, nitro, alkylthio, ethylenedioxy, aryl or benzyloxy groups, or R1 is a 1,3a-dihydropyrenylmethyl.

In some embodiments, R1 is benzyl substituted with one or more halo, alkoxy or hydroxy groups. In some embodiments, R1 is substituted with a halo and an alkoxy group.

In some embodiments, the compound of Formula (III) has a 1R,9S configuration.

Non-limiting example of compounds of Formula (III) useful for the purposes disclosed herein include:

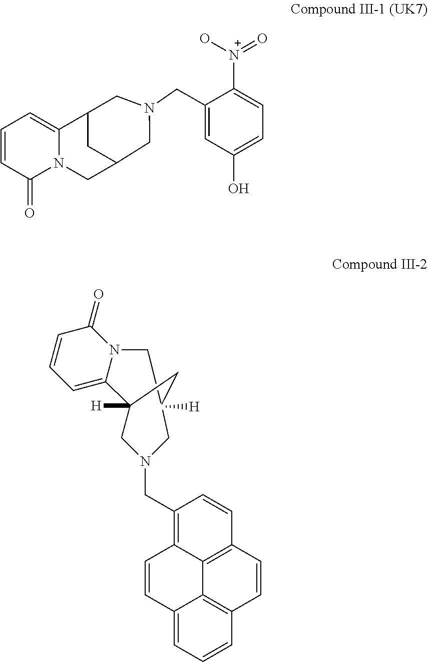

In some embodiments, the compound of Formula (III) is 11l-(5-hydroxy-2-nitrobenzyl)-7,11-diazatricyclo[7.3.1.0˜2,7˜]trideca-2,4-dien-6-one.

Methods for making compounds of Formula (III) may be found in US-2003065173-A1.

The disclosure is also directed to a compound of Formula (III) and pharmaceutical compositions comprising a compound of Formula (III). Non-limiting examples include Compounds III-1 through III-2.

In some embodiments, a compound is provided of Formula (IV):

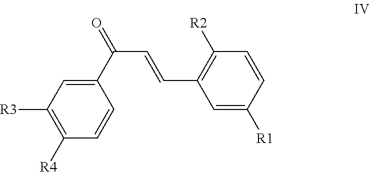

wherein R1 is halo or alkyloxy;
R2 and R3 are independently alkyloxy; and
R4 is hydrogen, alkyl or alkoxy.

In some embodiments, R1 is bromo or methoxy.

In some embodiments, R2 is methoxy or ethoxy.

In some embodiments, R3 is methoxy.

In some embodiments, R4 is methyl or methoxy.

Non-limiting examples of compounds of Formula (IV) useful for the purposes disclosed herein include:

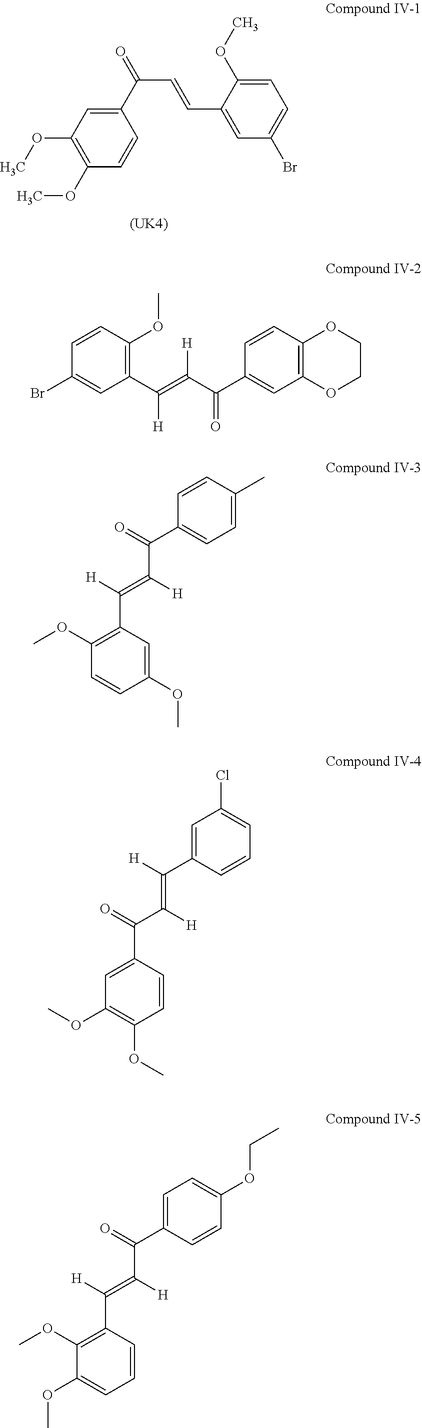

In some embodiments, the compound of Formula (IV) is 3-(5-bromo-2-methoxyphenyl)-1-(3,4-dimethoxyphenyl)-2-propen-1-one.

Compounds of formula (IV) may be prepared following the guidance in Puxeddu et al., European J Med Chem 2020; 185:111828; Meng et al., Bioorg Med Chem Letts 2004; 14:1513-1517; US-2016251312-A1; and US-2014329674-A1.

The disclosure is also directed to a compound of Formula (IV) and pharmaceutical compositions comprising a compound of Formula (IV). Non-limiting examples include Compounds IV-1 through IV-5.

As used herein, in some embodiments, the term “alkyl” refers to a saturated hydrocarbon group which is straight-chained or branched. Example alkyl groups include methyl (Me), ethyl (Et), propyl (e.g., n-propyl and isopropyl), butyl (e.g., n-butyl, isobutyl, t-butyl), pentyl (e.g., n-pentyl, isopentyl, neopentyl), and the like. An alkyl group can contain from 1 to about 20, from 2 to about 20, from 1 to about 10, from 1 to about 8, from 1 to about 6, from 1 to about 4, or from 1 to about 3 carbon atoms.

As used herein, “alkyloxy” or “alkoxy” refers to an —O-alkyl group.

In some embodiments, “aryl” refers to monocyclic or polycyclic (e.g., having 2, 3 or 4 fused rings) aromatic hydrocarbons such as, for example, phenyl, naphthyl, anthracenyl, phenanthrenyl, and the like. In some embodiments, an aryl group has from 6 to about 20 carbon atoms. In some embodiments, “aryl” may be optionally substituted at any one or more positions.

In some embodiments, “halo” or “halogen” includes fluoro, chloro, bromo, and iodo. A “halogen-substitution” or “halo” substitution designates replacement of one or more hydrogen atoms with F, Cl, Br or I.

It is understood that each of alkyl and aryl may be optionally substituted with independently selected groups such as alkyl, haloalkyl, hydroxyalkyl, aminoalkyl, carboxylic acid and derivatives thereof, including esters, amides, and nitrites, hydroxy, alkyloxy, acyloxy, amino, alky and dialkylamino, acylamino, thio, and the like, and combinations thereof.

In some embodiments, the term “substituted” refers to the replacement of a hydrogen moiety with a non-hydrogen moiety in a molecule or group. It can refer to “mono-substituted” or “poly-substituted.” The term “mono-substituted” or “poly-substituted” means substituted with one or more than one substituent up to the valence of the substituted group. For example, a mono-substituted group can be substituted with 1 substituent, and a poly-substituted group can be substituted with 2, 3, 4, or 5 substituents. When a list of possible substituents is provided, the substituents can be independently selected from that group.

The term “optionally substituted,” in some embodiments, refers to that the groups in question are either unsubstituted or substituted with one or more of the substituents specified. When the groups in question are substituted with more than one substituent, the substituents may be the same or different. Such other functional groups illustratively include, but are not limited to, amino, hydroxyl, CN, halo, thiol, alkyl, haloalkyl, heteroalkyl, aryl, arylalkyl, arylheteroalkyl, heteroaryl, heteroarylalkyl, heteroarylheteroalkyl, nitro, sulfonic acids and derivatives thereof, carboxylic acids and derivatives thereof, and the like. Illustratively, any of amino, hydroxyl, CH, thiol, alkyl, haloalkyl, heteroalkyl, aryl, arylalkyl, arylheteroalkyl, heteroaryl, heteroarylalkyl, heteroarylheteroalkyl, and/or sulfonic acid is optionally substituted. In some embodiments, the functional groups are the substituents described herein for any one of variables. Furthermore, when using the terms “independently,” “independently are,” and “independently selected from” mean that the groups in question may be the same or different. Certain of the herein defined terms may occur more than once in the structure, and upon such occurrence each term shall be defined independently of the other.

In each of the foregoing and each of the following embodiments, it is to be understood that the formulas also include any and all hydrates and/or solvates of the compound formulas. It is appreciated that certain functional groups, such as the hydroxy, amino, and like groups form complexes and/or coordination compounds with water and/or various solvents, in the various physical forms of the compounds. Accordingly, the above formulas are to be understood to include and represent those various hydrates and/or solvates.

Compounds disclosed herein can also include all isotopes of atoms occurring in the intermediates or final compounds. Isotopes include those atoms having the same atomic number but different mass numbers. For example, isotopes of hydrogen include tritium and deuterium.

Compounds described herein may contain one or more asymmetric centers and may thus give rise to diastereomers and optical isomers. The present disclosure includes all such possible optical isomers, diastereomers as well as their racemic mixtures, their substantially pure resolved enantiomers, all possible geometric isomers, and pharmaceutically acceptable salts thereof. The above Formula (I) is shown without a definitive stereochemistry at certain positions. The present disclosure includes all stereoisomers of Formula (I) and pharmaceutically acceptable salts thereof. Further, mixtures of stereoisomers as well as isolated specific stereoisomers are also included.

In some embodiments, the phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The present disclosure also includes “pharmaceutically acceptable salts” of the compounds described herein. As used herein, “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the compounds disclosed herein include the conventional non-toxic salts of the parent compound formed, for example, from non-toxic inorganic or organic acids. The pharmaceutically acceptable salts of the compounds disclosed herein can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred.

The pharmaceutically acceptable salts of the compounds disclosed herein can be also obtained by converting derivatives which possess tertiary amino groups into the corresponding quaternary ammonium salts in a manner known per se using quaternizing agents. Examples of suitable quaternizing agents are alkyl halides, such as methyl iodide, ethyl bromide, and n-propyl chloride, and also arylalkyl halides, such as benzyl chloride or 2-phenylethyl bromide. In some embodiments, the salts may be formed by conventional means, such as by reacting the free base or free acid form of the product with one or more equivalents of the appropriate acid or base in a solvent or medium in which the salt is insoluble or in a solvent such as water, which is removed in vacuo or by freeze drying or by exchanging the ions of an existing salt for another ion or suitable ion-exchange resin.

Possible pharmaceutically acceptable salts are well known in the art. For example, S. M. Berge, et al. describe pharmaceutically acceptable salts in detail in J. Pharmaceutical Sciences, 66: 1-19, 1977; incorporated herein by reference.

Typically, a pharmaceutically acceptable salt form of a compound can be prepared in situ during the final isolation and purification of the compound, or separately by reacting the free base functionality with a suitable organic or inorganic acid. Examples of typical pharmaceutically acceptable, nontoxic acid addition salts are salts of an amino group formed with inorganic acids such as hydrochloric acid, hydrobromic acid, phosphoric acid, sulfuric acid, and perchloric acid, or with organic acids such as acetic acid, oxalic acid, maleic acid, tartaric acid, citric acid, succinic acid, or malonic acid or by using other methods used in the art such as ion exchange.

Other pharmaceutically acceptable salts can include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hernisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and quaternary ammonium salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

This disclosure further includes derivatives of the compounds disclosed herein. The term “derivatives” includes but is not limited to ether derivatives, acid derivatives, amide derivatives, ester derivatives and the like. In addition, this disclosure further includes hydrates or solvates of the compounds disclosed herein. The term “hydrate” includes but is not limited to hemihydrate, monohydrate, dihydrate, trihydrate and the like.

In one embodiment, a compound is provided having Formula (I):

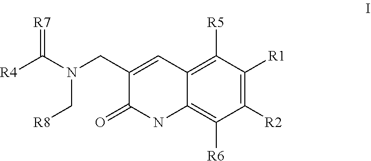

wherein R1 and R2 are independently hydrogen, alkyl, or alkyloxy; or R1 and R2 taken together is methylenedioxy or ethylenedioxy;
R8 is thienyl, furyl or 4-halophenyl;
R4 is 4-alkylphenyl, 2,4-dialkoxyphenyl, 4-alkylphenylamino, 4-alkoxyphenylamino, benzylamino, furylalkylamino or thienyl;
R5 and R6 are independently hydrogen or alkyl;
and R7 is sulfur or oxygen.

In some embodiments, R1 and R2 are independently hydrogen, ethoxy, methoxy or methyl. n some embodiments, R1 and R2 taken together is ethylenedioxy. In some embodiments, R8 is 2-thienyl, 2-furyl or 4-fluorophenyl. In some embodiments, R4 is 4-methylphenyl, 4-t-butyl-phenyl, 2,4-dimethoxyphenyl, 4-methylphenylamino, 4-ethoxyphenylamino, benzylamino, furylmethylamino or thienyl. In some embodiments, furylmethylamino is 2-furylmethylamino. In some embodiments, R5 and R6 are independently hydrogen or methyl. In some embodiments, R7 is sulfur or oxygen.

In some embodiments, a compound is provided having Formula (II):

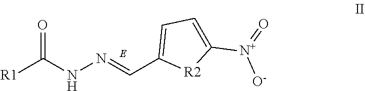

wherein R1 is aryl optionally substituted by one or more halo, alkyl or arylalkyl; and
R2 is oxygen or sulfur.

In some embodiments, R1 is phenyl optionally substituted with one or more halo or alkyl. In some embodiments, R1 is 4-halophenyl. In some embodiments, R1 is 3-halophenyl. In some embodiments, R1 is 4-(1,1′-biphenyl). In some embodiments, R1 is naphthyl. In some embodiments, R1 is 1-naphthyl. In some embodiments, R1 is phenylalkyl. In some embodiments, R1 is phenylethyl. In some embodiments, R1 is naphthylalkyl. In some embodiments, R1 is 1-naphthylmethyl.

In some embodiments, a compound of Formula (III):

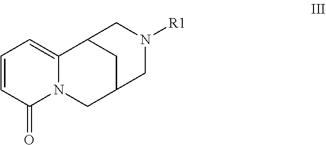

wherein R1 is benzyl optionally substituted with one or more halo, alkoxy, hydroxy, nitro, alkylthio, ethylenedioxy, aryl or benzyloxy groups, or R1 is a 1,3a-dihydropyrenylmethyl.

In some embodiments, R1 is benzyl substituted with one or more halo, alkoxy or hydroxy groups. In some embodiments, R1 is substituted with a halo and an alkoxy group. In some embodiments, the compound of Formula (III) has a 1R,9S configuration.

In some embodiments, a compound is provided of Formula (IV):

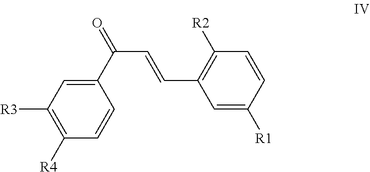

wherein R1 is halo or alkyloxy;
R2 and R3 are independently alkyloxy; and
R4 is hydrogen, alkyl or alkoxy.

In some embodiments, R1 is bromo or methoxy. In some embodiments, R2 is methoxy or ethoxy. In some embodiments, R3 is methoxy. In some embodiment, R4 is methyl or methoxy.

In some embodiments, pharmaceutical compositions comprising a compound of any one of Formulas (I)-(IV) is provided herein.

The present description provides methods and agents for treating an inflammatory disease in a live animal such as a human. In an aspect, the present description provides a method of treating an inflammatory disease in a live animal, the method of treatment comprising administering a therapeutically effective dose of a selective Il12b inhibitor, wherein the selective Il12b inhibitor may be selected from the list of compounds consisting of: 1-(2-nitro-1-propen-1-yl)naphthalene; 2-{[(5-nitro-2-thienyl)methylene]amino}benzamide; 1-ethoxy-4-(2-nitrovinyl)benzene; 3-(5-bromo-2-methoxyphenyl)-1-(3,4-dimethoxyphenyl)-2-propen-1-one; 2-(1H-benzimidazol-2-yl)-1-phenyl-3-(3-pyridinyl)-2-propen-1-one; 5,6-dimethyl-N-[(5-nitro-2-furyl)methylene]-1H-benzimidazol-1-amine; 11-(5-hydroxy-2-nitrobenzyl)-7,11-diazatricyclo[7.3.1.0˜2,7˜]trideca-2,4-dien-6-one; 6-chloro-2-(trichloromethyl)-4H-chromen-4-one; N-(5-bromo-2-biphenylyl)acetamide; diethyl [(1,3-dioxo-2,3-dihydro-1H-inden-5-yl)methylene]malonate; 5-ethylphenazin-5-ium ethyl sulfate; 1,2-dimethoxy-4-[(E)-2-nitroethenyl]benzene; 4-{[(1E,2E)-3-(5-nitrofuran-2-yl)prop-2-en-1-ylidene]amino}-5-phenyl-4H-1,2,4-triazole-3-thiol; (E)-1-ethoxy-2-(2-nitroprop-1-en-1-yl)benzene; (5E)-1-(3,4-Dimethylphenyl)-5-{[1-(4-methoxyphenyl)-1H-pyrrol-2-yl]methylidene}-2-thioxodihydropyrimidine-4,6(1H,5H)-dione; (1E,4E)-1,5-Bis(2-methoxyphenyl)penta-1,4-dien-3-one; 3-Fluoro-N′-[(E)-(5-nitrothiophen-2-yl)methylidene]benzohydrazide; 3-Methyl-5-(6-nitro-benzo[1,3]dioxol-5-ylmethylene)-2-thioxo-thiazolidin-4-one; 1-(2,3-Dihydro-benzo[1,4]dioxin-6-yl)-3-(3,4,5-trimethoxy-phenyl)-propenone; N-(2-Furylmethyl)-N-[(6-methoxy-2-oxo-1H-quinolin-3-yl)methyl]-4-methyl-benzamide; and N-[2-Chloro-5-(trifluoromethyl)phenyl]-5-nitrofuran-2-carboxamide. In preferred embodiments, the selective Il12b inhibitor may be selected from the list of compounds consisting of 1-(2-nitro-1-propen-1-yl)naphthalene; 2-{[(5-nitro-2-thienyl)methylene]amino}benzamide; 1-ethoxy-4-(2-nitrovinyl)benzene; 3-(5-bromo-2-methoxyphenyl)-1-(3,4-dimethoxyphenyl)-2-propen-1-one; 2-(1H-benzimidazol-2-yl)-1-phenyl-3-(3-pyridinyl)-2-propen-1-one; 5,6-dimethyl-N-[(5-nitro-2-furyl)methylene]-1H-benzimidazol-1-amine; 11-(5-hydroxy-2-nitrobenzyl)-7,11-diazatricyclo[7.3.1.0˜2,7˜]trideca-2,4-dien-6-one; 6-chloro-2-trichloromethyl)-4H-chromen-4-one; N-(5-bromo-2-biphenylyl)acetamide; diethyl [(1,3-dioxo-2,3-dihydro-1H-inden-5-yl)methylene]malonate; 5-ethylphenazin-5-ium ethyl sulfate; and 3-fluoro-N′-[(5-nitro-2-thienyl)methylene]benzohydrazide. In any embodiment, the selective Il12b inhibitor may comprise any combination of the above-listed compounds.

In an aspect, the present description provides a method of treating an inflammatory disease in a live animal, the method of treatment comprising administering a therapeutically effective dose of a selective Il12b inhibitor, wherein the selective Il12b inhibitor may be selected from any one of Formulas (I)-(IV), or any of the compounds disclosed herein.

Chemical structures for these compounds are as follows: 1-(2-nitro-1-propen-1-yl)naphthalene

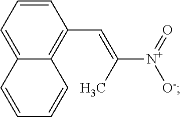

2-{[(5-nitro-2-thienyl)methylene]amino}benzamide

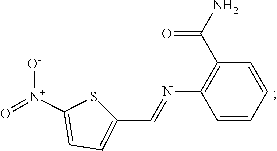 [SMILES: NC(=O)c1ccccc1/N=C/c1ccc([N+](=O)[O-])s1]

1-ethoxy-4-(2-nitrovinyl)benzene

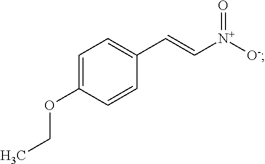

2-(1H-benzimidazol-2-yl)-1-phenyl-3-(3-pyridinyl)-2-propen-1-one

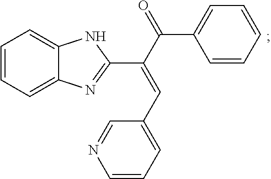 [SMILES: O=C(/C(=C/c1cccnc1)c1nc2ccccc2[nH]1)c1ccccc1]

5,6-dimethyl-N-[(5-nitro-2-furyl)methylene]-1H-benzimidazol-1-amine

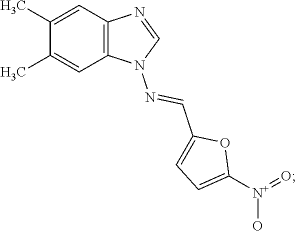

6-chloro-2-(trichloromethyl)-4H-chromen-4-one

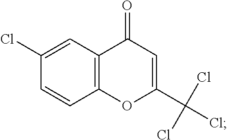 [SMILES: O=c1cc(C(Cl)(Cl)Cl)oc2ccc(Cl)cc12]

N-(5-bromo-2-biphenylyl)acetamide

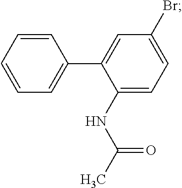 [SMILES: CC(=O)Nc1ccc(Br)cc1-c1ccccc1]

diethyl [(1,3-dioxo-2,3-dihydro-1-inden-5-yl)methylene]malonate

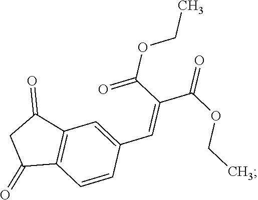 [SMILES: CCOC(=O)C(=Cc1ccc2c(c1)C(=O)CC2=O)C(=O)OCC]

5-ethylphenazin-5-ium ethyl sulfate

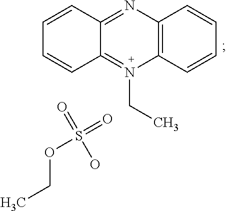

1,2-dimethoxy-4-[(E)-2-nitroethenyl]benzene

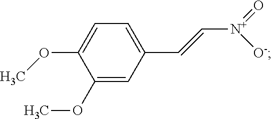

4-{[(1E,2E)-3-(5-nitrofuran-2-yl)prop-2-en-1-ylidene]amino}-5-phenyl-4H-1,2,4-triazole-3-thiol

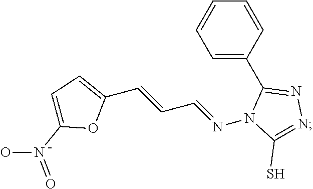

(E)-1-ethoxy-2-(2-nitroprop-1-en-1-yl)benzene

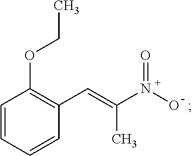

(5E)-1-(3,4-Dimethylphenyl)-5-{[1-(4-methoxyphenyl)-1H-pyrrol-2-yl]methylidene}-2-thioxodihydropyrimidine-4,6(1H,5H)-dione

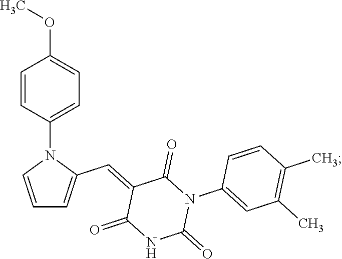 [SMILES: COc1ccc(-n2cccc2/C=C2\C(=O)NC(=O)N(c3ccc(C)c(C)c3)C2=O)cc1]

(1E,4E)-1,5-Bis(2-methoxyphenyl)penta-1,4-dien-3-one

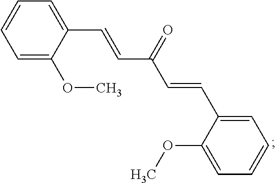 [SMILES: COc1ccccc1/C=C/C(=O)/C=C/c1ccccc1OC]

3-Methyl-5-(6-nitro-benzo[1,3]dioxol-5-ylmethylene)-2-thioxo-thiazolidin-4-one

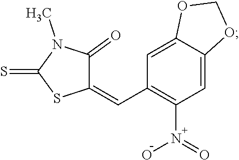

1-(2,3-Dihydro-benzo[1,4]dioxin-6-yl)-3-(3,4,5-trimethoxy-phenyl)-propenone

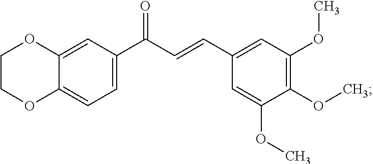 [SMILES: COc1cc(/C=C/C(=O)c2ccc3c(c2)OCCO3)cc(OC)c1OC]

and N-[2-Chloro-5-(trifluoromethyl)phenyl]-5-nitrofuran-2-carboxamide

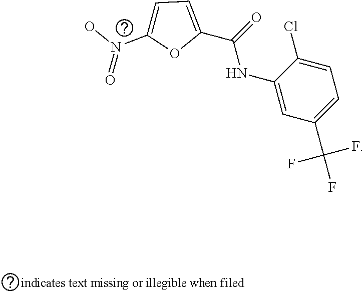

It will be readily understood to persons skilled in the art that the chemical names for these compounds may be one of several acceptable alternative chemical names. Likewise, it will be readily understood to persons skilled in the art that a chemical structure may represent one of several interchangeable conformations, isomers, tautomers, and the like.

In another aspect, the present description provides a method of treatment of an inflammatory disease in a live animal such as a human, the method comprising administering a therapeutically effective dose of a selective Il12b inhibitor, wherein the selective Il12b inhibitor is a Gα G-protein coupled receptor (GPCR) activator.

In another aspect, the present description provides a method of treatment of an inflammatory disease in a live animal such as a human, the method comprising administering a therapeutically effective dose of a selective Il12b inhibitor, wherein the selective Il12b inhibitor is a Gαs-subtype GPCR activator.

In another aspect, the present description provides a method of treatment of an inflammatory disease in a live animal such as a human, the method comprising administering a therapeutically effective dose of a selective Il12b inhibitor, wherein the selective Il12b inhibitor is a Gαi/o-subtype GPCR activator.

In another aspect, the present description provides a method of treatment an inflammatory disease in a live animal such as a human, the method comprising administering a therapeutically effective dose of a selective Il12b inhibitor, wherein the selective Il12b inhibitor is a Gαq/11-subtype GPCR activator.

In another aspect, the present description provides a method of treatment an inflammatory disease in a live animal such as a human, the method comprising administering a therapeutically effective dose of a selective Il12b inhibitor, wherein the selective Il12b inhibitor is a Gαl2/13-subtype GPCR activator.

In yet another aspect, the present description provides a method of treating an inflammatory disease in a live animal such as a human, the method comprising administering a therapeutically effective dose of a selective Il12b inhibitor, wherein the selective Il12b inhibitor is a β adrenergic activator. In any embodiment, the β adrenergic activator may be a β adrenergic agonist. In such embodiments, the β adrenergic agonist is may be selected from the group consisting of: denopamine, dobutamine, dopexamine, epinephrine, isoprenaline, isoproterenol, prenalterol, xamoterol, arformoterol, buphenine, clenbuterol, fenoterol, formoterol, isoetarine, levosalbutamol, levalbuterol, orciprenaline, metaproterenol, pirbuterol, procaterol, ritodrine, salbutamol, albuterol, salmeterol, and terbutaline. In any embodiment, the β adrenergic agonist may comprise any combination of the above-listed compounds.

In still another aspect the present description provides a method of treating an inflammatory disease in a live animal such as a human, the method comprising administering a therapeutically effective dose of a selective Il12b inhibitor, wherein the selective Il12b inhibitor is a β₂ adrenergic agonist. In any embodiment of the method of treatment, the β₂ adrenergic agonist may be selected from the list of compounds consisting of: arformoterol, buphenine, clenbuterol, dopexamine, epinephrine, fenoterol, formoterol, isoetarine. isoprenaline, isoproterenol, levosalbutamol, levalbuterol, orciprenaline, metaproterenol, pirbuterol, procaterol, ritodrine, salbutamol, albuterol, salmeterol, and terbutaline. In any embodiment, the β₂ adrenergic agonist may comprise any combination of the above-listed compounds.

The method of treating inflammatory disease may comprise administering a therapeutically effective dose of prostanoid receptor agonist causing an increase in intracellular cAMP formation. The prostanoid receptors may comprise EP(2), EP(4), or a combination thereof. The prostanoid receptor agonist may comprise PGE2. The prostanoid receptor agonist may be combined with any another therapeutic or compound disclosed in the present description, such as a compound of any of Formulas (I)-(IV), any other compound disclosed herein, or a β adrenergic agonist or a β₂ adrenergic agonist.

In yet another aspect, the present description provides a method of treating an inflammatory disease in a live animal such as a human comprising administering a therapeutically effective dose of a combination of: (a) a composition comprising a Gα protein coupled receptor activator that activates cyclic AMP signaling; and (b) composition comprising a selective inhibitor of cyclic AMP (cAMP) breakdown. In any embodiment of the method of treatment, the Gα protein coupled receptor activator that activates cyclic AMP signaling may be any of a Gα GPCR activator, Gαs-subtype GPCR activator, Gαi/o-subtype GPCR activator, Gαq/11-subtype GPCR activator, Gαl2/13-subtype GPCR activator, β adrenergic activator, β adrenergic agonist, β₂ adrenergic activator or a β₂ adrenergic agonist. In any embodiment, the inhibitor of cyclic AMP breakdown is a phosphodiesterase 4 inhibitor (PDE4 inhibitor). In any embodiment, the PDE4 inhibitor may be a selective PDE4 inhibitor. In any embodiment, the PDE4 inhibitor may be selected from the group consisting of mesembrenone, rolipram, ibudilast, piclimilast, luteolin, drotaverine, roflumilast, crisaborole, apremilast, and any combination thereof. In any embodiment of the method of treating inflammatory disease, each one or both components may be administered topically in the form of a cream, salve, gel, dermal patch, aqueous solution, or non-aqueous solution. In some embodiments, the PDE4 inhibitor is administered topically or systemically, such as orally. In some embodiments, the PDE4 inhibitor is administered topically with the GPCR activator, or administered systemically, such as orally.

In yet another aspect, the present description provides a method of treating an inflammatory disease in a live animal comprising administering a therapeutically effective dose of a combination of:
(a) a compound of any one of Formulas (I)-(IV) or a compound selected from among 1-(2-nitro-1-propen-1-yl)naphthalene; 2-{[(5-nitro-2-thienyl)methylene]amino}benzamide; 1-ethoxy-4-(2-nitrovinyl)benzene; 3-(5-bromo-2-methoxyphenyl)-1-(3,4-dimethoxyphenyl)-2-propen-1-one; 2-(1H-benzimidazol-2-yl)-1-phenyl-3-(3-pyridinyl)-2-propen-1-one; 5,6-dimethyl-N-[(5-nitro-2-furyl)methylene]-1H-benzimidazol-1-amine; 11-(5-hydroxy-2-nitrobenzyl)-7,11-diazatricyclo[7.3.1.0˜2,7˜]trideca-2,4-dien-6-one; 6-chloro-2-(trichloromethyl)-4H-chromen-4-one; N-(5-bromo-2-biphenylyl)acetamide; diethyl [(1,3-dioxo-2,3-dihydro-1H-inden-5-yl)methylene]malonate; 5-ethylphenazin-5-ium ethyl sulfate; 1,2-dimethoxy-4-[(E)-2-nitroethenyl]benzene; 4-{[(1E,2E)-3-(5-nitrofuran-2-yl)prop-2-en-1-ylidene]amino}-5-phenyl-4H-1,2,4-triazole-3-thiol; (E)-1-ethoxy-2-(2-nitroprop-1-en-1-yl)benzene; (5E)-1-(3,4-Dimethylphenyl)-5-{[1-(4-methoxyphenyl)-1H-pyrrol-2-yl]methylidene}-2-thioxodihydropyrimidine-4,6(1H,5H)-dione; (1E,4E)-1,5-Bis(2-methoxyphenyl)penta-1,4-dien-3-one; 3-Fluoro-N′-[(E)-(5-nitrothiophen-2-yl)methylidene]benzohydrazide; 3-Methyl-5-(6-nitro-benzo[1,3]dioxol-5-ylmethylene)-2-thioxo-thiazolidin-4-one; 1-(2,3-Dihydro-benzo[1,4]dioxin-6-yl)-3-(3,4,5-trimethoxy-phenyl)-propenone; N-(2-Furylmethyl)-N-[(6-methoxy-2-oxo-1H-quinolin-3-yl)methyl]-4-methyl-benzamide; and N-[2-Chloro-5-(trifluoromethyl)phenyl]-5-nitrofuran-2-carboxamide; and
(b) composition comprising a selective inhibitor of cyclic AMP (cAMP) breakdown.

In any embodiment, the inhibitor of cyclic AMP breakdown is a phosphodiesterase 4 inhibitor (PDE4 inhibitor).

In any embodiment, the PDE4 inhibitor may be a selective PDE4 inhibitor. In any embodiment, the PDE4 inhibitor may be selected from the group consisting of mesembrenone, rolipram, ibudilast, piclimilast, luteolin, drotaverine, roflumilast, crisaborole, apremilast, and any combination thereof.

In any embodiment of the method, each one or both components may be administered topically in the form of a cream, salve, gel, dermal patch, aqueous solution, or non-aqueous solution. In some embodiments, the PDE4 inhibitor is administered topically or systemically, such as orally. In some embodiments, the PDE4 inhibitor is administered topically with the GPCR activator, or administered systemically, such as orally.

In one aspect, a therapeutic combination is provided comprising any one or more of the IL12b inhibitors disclosed herein, and any one or more inhibitor of cAMP breakdown. In some embodiments, the therapeutic composition is a formulation comprising both one or more IL12b inhibitors disclosed herein, and one or more inhibitors of cAMP breakdown. In other embodiments, the one or more IL12b inhibitors disclosed herein, and one or more inhibitors of cAMP breakdown, are in separate formulations, but administered concurrently or in an overlapping schedule. In some embodiments, the components are administered by the same route of administration or by different routes of administration. In one embodiment, the IL12b inhibitor of the therapeutic combination is administered topically, and the inhibitor of cAMP breakdown is administered topically or systemically, such as orally.

In some embodiments, the therapeutic combination is a synergistic combination, for example, the effectiveness achieved by administration of the combination is greater than the combined effects of the two components administered at the doses or dose regimens in the combination but in the absence of the other component. Such synergistic therapeutic combination does not necessarily comprise a single formulation comprising both components, but a dosing regimen of each components that produces a synergistic effect.

Thus, in one embodiment, a therapeutic combination of an IL12b inhibitor such as but not limited to those described herein, and an inhibitor of cAMP breakdown such as but not limited to those described herein, elicits greater effectiveness than achieved by either the IL12b inhibitor administered alone or the inhibitor of cAMP breakdown when administered alone.

This, in one embodiment, the IL12b inhibitor component of the therapeutic combination may be any one or more of a compound of any one or more of Formulas (I)-(IV); 1-(2-nitro-1-propen-1-yl)naphthalene; 2-{[(5-nitro-2-thienyl)methylene]amino}benzamide; 1-ethoxy-4-(2-nitrovinyl)benzene; 3-(5-bromo-2-methoxyphenyl)-1-(3,4-dimethoxyphenyl)-2-propen-1-one; 2-(1H-benzimidazol-2-yl)-1-phenyl-3-(3-pyridinyl)-2-propen-1-one; 5,6-dimethyl-N-[(5-nitro-2-furyl)methylene]-1H-benzimidazol-1-amine; 11-(5-hydroxy-2-nitrobenzyl)-7,11-diazatricyclo[7.3.1.0˜2,7˜]trideca-2,4-dien-6-one; 6-chloro-2-(trichloromethyl)-4H-chromen-4-one; N-(5-bromo-2-biphenylyl)acetamide; diethyl [(1,3-dioxo-2,3-dihydro-1H-inden-5-yl)methylene]malonate; 5-ethylphenazin-5-ium ethyl sulfate; 1,2-dimethoxy-4-[(E)-2-nitroethenyl]benzene; 4-{[(1E,2E)-3-(5-nitrofuran-2-yl)prop-2-en-1-ylidene]amino}-5-phenyl-4H-1,2,4-triazole-3-thiol; (E)-1-ethoxy-2-(2-nitroprop-1-en-1-yl)benzene; (5E)-1-(3,4-Dimethylphenyl)-5-{[1-(4-methoxyphenyl)-1H-pyrrol-2-yl]methylidene}-2-thioxodihydropyrimidine-4,6(1H,5H)-dione; (1E,4E)-1,5-Bis(2-methoxyphenyl)penta-1,4-dien-3-one; 3-Fluoro-N′-[(E)-(5-nitrothiophen-2-yl)methylidene]benzohydrazide; 3-Methyl-5-(6-nitro-benzo[1,3]dioxol-5-ylmethylene)-2-thioxo-thiazolidin-4-one; 1-(2,3-Dihydro-benzo[1,4]dioxin-6-yl)-3-(3,4,5-trimethoxy-phenyl)-propenone; N-(2-Furylmethyl)-N-[(6-methoxy-2-oxo-1H-quinolin-3-yl)methyl]-4-methyl-benzamide; and N-[2-Chloro-5-(trifluoromethyl)phenyl]-5-nitrofuran-2-carboxamide, or an analogue of any of the foregoing, or β adrenergic agonist. The compound of any one of Formulas (I)-(IV) may be N-(2-furylmethyl)-N-[(6-methoxy-2-oxo-1H-quinolin-3-yl)methyl]-4-methyl-benzamide; N-[(5,8-dimethyl-2-oxo-1H-quinolin-3-yl)methyl]-N-[(4-fluorophenyl)methyl]-3,5-dimethoxybenzamide; 4-tert-butyl-N-[(6-ethoxy-2-oxo-1H-quinolin-3-yl)methyl]-N-(furan-2-ylmethyl)benzamide; 1-(furan-2-ylmethyl)-3-(4-methylphenyl)-1-[(7-oxo-3,6-dihydro-2H-[1,4]dioxino[2,3-g]quinolin-8-yl)methyl]urea; 1-[(6-ethoxy-2-oxo-1H-quinolin-3-yl)methyl]-1-(furan-2-ylmethyl)-3-(4-methoxyphenyl)urea; 3-(4-ethoxyphenyl)-1-(furan-2-ylmethyl)-1-[(7-methyl-2-oxo-1H-quinolin-3-yl)methyl]thiourea; 1-(furan-2-ylmethyl)-3-(4-methylphenyl)-1-[(6-oxo-5H-[1,3]dioxolo[4,5-g]quinolin-7-yl)methyl]urea; 3-benzyl-1-(furan-2-ylmethyl)-1-[(6-oxo-5H-[1,3]dioxolo[4,5-g]quinolin-7-yl)methyl]urea; 3-benzyl-1-[(6-ethyl-2-oxo-1H-quinolin-3-yl)methyl]-1-(furan-2-ylmethyl)thiourea; 1-[(5,8-dimethyl-2-oxo-1H-quinolin-3-yl)methyl]-1,3-bis(furan-2-ylmethyl)thiourea; N-[(6-ethoxy-2-oxo-1H-quinolin-3-yl)methyl]-N-(furan-2-ylmethyl)thiophene-2-carboxamide, Non-limiting example of compounds of Formula (II) include 3-fluoro-N′-[(5-nitro-2-thienyl)methylene]benzohydrazide; 5,6-dimethyl-N-[(5-nitro-2-furyl)methylene]-1H-benzimidazol-1-amine; N—[(Z)-(5-nitrothiophen-2-yl)methylideneamino]-3-phenylpropanamide; II-3: N—[(Z)-(5-nitrofuran-2-yl)methylideneamino]-4-phenylbenzamide; II-4: 3-chloro-N-[(E)-1-(5-nitrofuran-2-yl)ethylideneamino]benzamide; 2-(4-chlorophenyl)-N—[(Z)-(5-nitrofuran-2-yl)methylideneamino]acetamide; and N—[(Z)-(5-nitrofuran-2-yl)methylideneamino]naphthalene-1-carboxamide, 11-(5-hydroxy-2-nitrobenzyl)-7,11-diazatricyclo[7.3.1.0˜2,7˜]trideca-2,4-dien-6-one, (1R,9S)-11-(pyren-1-ylmethyl)-7,11-diazatricyclo[7.3.1.0²⁷]trideca-2,4-dien-6-one, 3-(5-bromo-2-methoxyphenyl)-1-(3,4-dimethoxyphenyl)-2-propen-1-one; (E)-3-(5-bromo-2-methoxyphenyl)-1-(2,3-dihydro-1,4-benzodioxin-6-yl)prop-2-en-1-one; (E)-3-(2,5-dimethoxyphenyl)-1-(4-methylphenyl)prop-2-en-1-one; (E)-3-(3-chlorophenyl)-1-(3,4-dimethoxyphenyl)prop-2-en-1-one; or (E)-3-(2,3-dimethoxyphenyl)-1-(4-ethoxyphenyl)prop-2-en-1-one, and any combination thereof.

The IL12b inhibitor component of the therapeutic combination may be a β adrenergic activator. In any embodiment, the β adrenergic activator may be a β adrenergic agonist. In such embodiments, the β adrenergic agonist may be selected from the group consisting of: denopamine, dobutamine, dopexamine, epinephrine, isoprenaline, isoproterenol, prenalterol, xamoterol, arformoterol, buphenine, clenbuterol, fenoterol, formoterol, isoetarine, levosalbutamol, levalbuterol, orciprenaline, metaproterenol, pirbuterol, procaterol, ritodrine, salbutamol, albuterol, salmeterol, and terbutaline. In some embodiments, the selective Il12b inhibitor of the therapeutic combination is a β₂ adrenergic activator or a β₂ adrenergic agonist, such as from among arformoterol, buphenine, clenbuterol, dopexamine, epinephrine, fenoterol, formoterol, isoetarine, isoprenaline, isoproterenol, levosalbutamol, levalbuterol, orciprenaline, metaproterenol, pirbuterol, procaterol, ritodrine, salbutamol, albuterol, salmeterol, and terbutaline.

The therapeutic combination component that inhibits the breakdown of cAMP may be, in one embodiment, a phosphodiesterase 4 inhibitor (PDE4 inhibitor), such as a selective PDE4 inhibitor, such as but not limited to mesembrenone, rolipram, ibudilast, piclimilast, luteolin, drotaverine, roflumilast, crisaborole, apremilast, and any combination thereof.

In any aspect or embodiment, the live animal may be a mammal. In any such aspect or embodiment, the mammal may be a human.

In any aspect or embodiment, the inflammatory disease may be any of psoriasis, chronic eczema, vitiligo, lichen planus, cutaneous lupus erythematosus, Behçet's disease, ulcerative colitis, Crohn's disease, or alopecia.

In any aspect or embodiment, the inflammatory disease may be an autoimmune disease. Non-limiting examples of autoimmune diseases include type 1 diabetes, rheumatoid arthritis, psoriasis and psoriatic arthritis, multiple sclerosis, systemic lupus erythematosus, inflammatory bowel disease including Crohn's disease and ulcerative colitis, Addison's disease, celiac disease, dermatomyositis, Grave's disease, Sjögren's syndrome, Hashimoto thyroiditis, autoimmune vasculitis, multiple sclerosis, myasthenia gravis, and pernicious anemia.

The dosing regimen, e.g., dose level, frequency of daily administration (e.g., once, twice, thrice daily), frequency of dosing, e.g., daily, every other day, weekly, etc., and duration of administration, e.g., chronically, or until stabilization or reversal of symptoms occur, and cycles of administration with periods of no administration, may be readily tailored to each individual subject by the medical professional.

Dose levels may be further determined or tailored according to appropriate clinical recommendation for stabilization or reversal of symptoms. Dose may be readily tailored to each individual subject by the medical professional.

In practice, the compounds disclosed herein, for example, represented by Formula (I)-(IV), other compounds disclosed herein, the β adrenergic agonists and/or the PDE4 inhibitors, or pharmaceutically acceptable salts thereof, can be combined as the active ingredient in intimate admixture with a pharmaceutical carrier according to conventional pharmaceutical compounding techniques. The carrier may take a wide variety of forms depending on the form of preparation desired for administration. e.g., oral or parenteral (including intravenous). Thus, the pharmaceutical compositions of the present disclosure can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil in water emulsion, or as a water in oil liquid emulsion.

In addition to the common dosage forms set out above, the compounds described herein, or a pharmaceutically acceptable salt thereof, may also be administered by controlled release means and/or delivery devices. The compositions may be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

Solid medicinal forms can comprise inert components and carrier substances, such as calcium carbonate, calcium phosphate, sodium phosphate, lactose, starch, mannitol, alginates, gelatine, guar gum, magnesium stearate, aluminum stearate, methyl cellulose, talc, highly dispersed silicic acids, silicone oil, higher molecular weight fatty acids, (such as stearic acid), gelatine, agar agar or vegetable or animal fats and oils, or solid high molecular weight polymers (such as polyethylene glycol); preparations which are suitable for oral administration can comprise additional flavorings and/or sweetening agents, if desired.

Liquid medicinal forms can be sterilized and/or, where appropriate, comprise auxiliary substances, such as preservatives, stabilizers, wetting agents, penetrating agents, emulsifiers, spreading agents, solubilizers, salts, sugars or sugar alcohols for regulating the osmotic pressure or for buffering, and/or viscosity regulators. Examples of such additives are tartrate and citrate buffers, ethanol and sequestering agents (such as ethylenediaminetetraacetic acid and its nontoxic salts). High molecular weight polymers, such as liquid polyethylene oxides, microcrystalline celluloses, carboxymethyl celluloses, polyvinylpyrrolidones, dextrans or gelatine, are suitable for regulating the viscosity. Examples of solid carrier substances are starch, lactose, mannitol, methyl cellulose, talc, highly dispersed silicic acids, high molecular weight fatty acids (such as stearic acid), gelatine, agar agar, calcium phosphate, magnesium stearate, animal and vegetable fats, and solid high molecular weight polymers, such as polyethylene glycol.

Oily suspensions for parenteral or topical applications can be vegetable synthetic or semisynthetic oils, such as liquid fatty acid esters having in each case from 8 to 22 C atoms in the fatty acid chains, for example palmitic acid, lauric acid, tridecanoic acid, margaric acid, stearic acid, arachidic acid, myristic acid, behenic acid, pentadecanoic acid, linoleic acid, elaidic acid, brasidic acid, erucic acid or oleic acid, which are esterified with monohydric to trihydric alcohols having from 1 to 6 C atoms, such as methanol, ethanol, propanol, butanol, pentanol or their isomers, glycol or glycerol. Examples of such fatty acid esters are commercially available miglyols, isopropyl myristate, isopropyl palmitate, isopropyl stearate, PEG 6-capric acid, caprylic/capric acid esters of saturated fatty alcohols, polyoxyethylene glycerol trioleates, ethyl oleate, waxy fatty acid esters, such as artificial ducktail gland fat, coconut fatty acid isopropyl ester, oleyl oleate, decyl oleate, ethyl lactate, dibutyl phthalate, diisopropyl adipate, polyol fatty acid esters, inter alia. Silicone oils of differing viscosity, or fatty alcohols, such as isotridecyl alcohol, 2-octyldodecanol, cetylstearyl alcohol or oleyl alcohol, or fatty acids, such as oleic acid, are also suitable. It is furthermore possible to use vegetable oils, such as castor oil, almond oil, olive oil, sesame oil, cotton seed oil, groundnut oil or soybean oil.

Suitable solvents, gelatinizing agents and solubilizers are water or water-miscible solvents. Examples of suitable substances are alcohols, such as ethanol or isopropyl alcohol, benzyl alcohol, 2-octyldodecanol, polyethylene glycols, phthalates, adipates, propylene glycol, glycerol, di- or tripropylene glycol, waxes, methyl cellosolve, cellosolve, esters, morpholines, dioxane, dimethyl sulphoxide, dimethylformamide, tetrahydrofuran, cyclohexanone, etc.

Mixtures of gelatinizing agents and film-forming agents are also perfectly possible. In this case, use is made, in particular, of ionic macromolecules such as sodium carboxymethyl cellulose, polyacrylic acid, polymethacrylic acid and their salts, sodium amylopectin semiglycolate, alginic acid or propylene glycol alginate as the sodium salt, gum arabic, xanthan gum, guar gum or carrageenan. The following can be used as additional formulation aids: glycerol, paraffin of differing viscosity, triethanolamine, collagen, allantoin and novantisolic acid. Use of surfactants, emulsifiers or wetting agents, for example of Na lauryl sulphate, fatty alcohol ether sulphates, di-Na—N-lauryl-β-iminodipropionate, polyethoxylated castor oil or sorbitan monooleate, sorbitan monostearate, polysorbates (e.g. Tween), cetyl alcohol, lecithin, glycerol monostearate, polyoxyethylene stearate, alkylphenol polyglycol ethers, cetyltrimethylammonium chloride or mono-/dialkylpolyglycol ether orthophosphoric acid monoethanolamine salts can also be required for the formulation. Stabilizers, such as montmorillonites or colloidal silicic acids, for stabilizing emulsions or preventing the breakdown of active substances such as antioxidants, for example tocopherols or butylhydroxyanisole, or preservatives, such as p-hydroxybenzoic acid esters, can likewise be used for preparing the desired formulations.

Preparations for parenteral administration can be present in separate dose unit forms, such as ampoules or vials. Use is preferably made of solutions of the active compound, preferably aqueous solution and, in particular, isotonic solutions and also suspensions. These injection forms can be made available as ready-to-use preparations or only be prepared directly before use, by mixing the active compound, for example the lyophilisate, where appropriate containing other solid carrier substances, with the desired solvent or suspending agent.

Intranasal preparations can be present as aqueous or oily solutions or as aqueous or oily suspensions. They can also be present as lyophilisates which are prepared before use using the suitable solvent or suspending agent.

Inhalable preparations can present as powders, solutions or suspensions. Preferably, inhalable preparations are in the form of powders, e.g. as a mixture of the active ingredient with a suitable formulation aid such as lactose.

The preparations are produced, aliquoted and sealed under the customary antimicrobial and aseptic conditions.

Thus, the present disclosure further provides a pharmaceutical composition comprising a compound disclosed herein as described herein, or a pharmaceutically acceptable salt thereof, and a pharmaceutically acceptable carrier.

The term “carrier” refers to any chemical entity that can be incorporated into a composition containing an active agent (any formula or compound disclosed herein) without interfering with the stability and/or activity of the agent. In some embodiments, the term “carrier” refers to a pharmaceutically acceptable carrier. An exemplary carrier herein is water.

As used herein, in some embodiments, “pharmaceutical composition” refers to therapeutically effective amounts of the compounds disclosed herein together with suitable diluents, preservatives, solubilizers, emulsifiers, adjuvant and/or carriers.

Also comprehended by the disclosure are particulate compositions coated with polymers (e.g., poloxamers or poloxamines). Other embodiments of the compositions disclosed herein incorporate particulate forms protective coatings, protease inhibitors or permeation enhancers for various routes of administration, including parenteral, pulmonary, nasal and oral. In some embodiments, the pharmaceutical composition is administered parenterally, paracancerally, transmucosally, transdermally, intramuscularly, intravenously, intradermally, subcutaneously, intraperitoneally, intraventricularly, intravaginally, intracranially and intratumorally.

As used herein, in some embodiments, the terms “therapeutically effective amount” and “effective amount” of an agent refer to an amount sufficient to provide a therapeutic benefit in the treatment, prevention and/or management of a disease, disorder, or condition, e.g., to delay onset of or minimize (e.g., reduce the incidence, frequency, and/or magnitude of) one or more symptoms associated with the disease, disorder or condition to be treated. Those of ordinary skill in the art will appreciate that, a composition may be said to contain a “therapeutically effective amount” of an agent if it contains an amount that is effective when administered as a single dose within the context of a therapeutic regimen. In some embodiments, a therapeutically effective amount is an amount that, when administered as part of a dosing regimen, is statistically likely to delay onset of or minimize (reduce the incidence and/or magnitude of) one or more symptoms or side effects of a disease, disorder or condition. In some embodiments, a “therapeutically effective amount” is an amount that enhances therapeutic efficacy of another agent with which the composition is administered in combination.

In some embodiments, a therapeutically effective amount for administration to a human corresponds to a reference amount (e.g., a therapeutically effective amount in an animal model such as a mouse model) adjusted for body surface area of a human as compared with body surface area of the animal model, as is known in the art (see, for example Reagan-Shaw et al., “Dose translation from animal to human studies revisited,” The FASEB Journal 22: 659-661 (2007), the entirety of which is herein incorporated by reference). In some embodiments, the reference therapeutically effective amount is an amount that is therapeutically effective in an animal model (e.g., in a mouse model). In some embodiments, the reference therapeutically effective amount is within the range of about 0.01 mg/kg to about 500 mg/kg. In some embodiments, the reference therapeutically effective amount is within the range of about 0.01 mg/kg to about 0.1 mg/kg. In some embodiments, the reference therapeutically effective amount is within the range of about 0.1 mg/kg to about 0.5 mg/kg. In some embodiments, the reference therapeutically effective amount is within the range of about 0.5 mg/kg to about 1 mg/kg. In some embodiments, the reference therapeutically effective amount is within the range of about 1 mg/kg to about 2.5 mg/kg. In some embodiments, the reference therapeutically effective amount is within the range of about 2.5 mg/kg to about 10 mg/kg. In some embodiments, the reference therapeutically effective amount is within the range of about 10 mg/kg to about 50 mg/kg. In some embodiments, the reference therapeutically effective amount is within the range of about 50 mg/kg to about 100 mg/kg. In some embodiments, the reference therapeutically effective amount is within the range of about 100 mg/kg to about 250 mg/kg. In some embodiments, the reference therapeutically effective amount is within the range of about 250 mg/kg to about 500 mg/kg.

In some embodiments, the term “comprise” or grammatical forms thereof, refers to the inclusion of the indicated active agent, such as the compound of this disclosure, as well as inclusion of other active agents, and pharmaceutically acceptable carriers, excipients, emollients, stabilizers, etc., as are known in the pharmaceutical industry. In some embodiments, the term “consisting essentially of” refers to a composition, whose only active ingredient is the indicated active ingredient, however, other compounds may be included which are for stabilizing, preserving, etc. the formulation, but are not involved directly in the therapeutic effect of the indicated active ingredient. In some embodiments, the term “consisting essentially of” may refer to components, which exert a therapeutic effect via a mechanism distinct from that of the indicated active ingredient. In some embodiments, the term “consisting essentially of” may refer to components, which exert a therapeutic effect and belong to a class of compounds distinct from that of the indicated active ingredient. In some embodiments, the term “consisting essentially of” may refer to components which facilitate the release of the active ingredient. In some embodiments, the term “consisting” refers to a composition, which contains the active ingredient and a pharmaceutically acceptable carrier or excipient.

In some embodiments, the pharmaceutically acceptable carriers, adjuvants, or vehicles refer to a non-toxic carrier, adjuvant, or vehicle that does not destroy the pharmacological activity of the compound with which it is formulated. Pharmaceutically acceptable carriers, adjuvants or vehicles that may be used in the compositions of this disclosure include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

As used herein, the term “combination,” “combined,” and related terms refer to the simultaneous exposure of a subject to two or more therapeutic agents in accordance with this disclosure. It will be appreciated that two or more agents are considered to be administered “in combination” whenever a subject is simultaneously exposed to both (or more) of the agents. Each of the two or more agents may be administered according to a different schedule; it is not required that individual doses of different agents be administered at the same time, or in the same composition. Rather, so long as both (or more) agents remain in the subject's body, they are considered to be administered “in combination.” For example, one or more doses of a compound of the present disclosure may be administered with another therapeutic agent simultaneously or sequentially in separate unit dosage forms or together in a single unit dosage form. Accordingly, in some embodiments, the present disclosure provides a single unit dosage form comprising a compound of formula (I), an additional therapeutic agent, and a pharmaceutically acceptable carrier, adjuvant, or vehicle.

The term “formulation” refers to a composition that includes at least one active agent (e.g., a compound as disclosed herein, or a compound of any of Formulas (I)-(IV) disclosed herein) in combination with one or more carriers, excipients or other pharmaceutical additives for administration to a patient. In general, particular carriers, excipients and/or other pharmaceutical additives are selected in accordance with knowledge in the art to achieve a desired stability, release, distribution and/or activity of active agent(s).

The term “parenteral” as used herein includes subcutaneous, intravenous, intramuscular, intra-articular, intra-synovial, intrastemal, intrathecal, intrahepatic, intralesional and intracranial injection or infusion techniques. Formulations provided herein for parenteral administration are typically water or oil-based (e.g., aqueous or oleaginous) suspensions or solutions. Such formulations may be prepared according to techniques known in the art, for example using suitable dispersing, wetting agents or suspending agents. In some embodiments, such formulations are sterile injectable solutions or suspensions in a non-toxic parenterally acceptable diluent or solvent, for example as a solution in 1,3-butanediol. Among the acceptable vehicles and solvents that may be employed are water, Ringer's solution and isotonic sodium chloride solution. Alternatively or additionally, sterile, fixed oils are conventionally employed as a solvent or suspending medium.

In some embodiments, the pharmaceutical compositions of the present disclosure can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil in water emulsion, or as a water in oil liquid emulsion. In addition to the common dosage forms set out above, the compound, or a pharmaceutically acceptable salt thereof, may also be administered by controlled release means and/or delivery devices. The compositions may be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

For example, a formulation intended for the oral administration to humans may contain from about 0.5 mg to about 5 g of active agent, compounded with an appropriate and convenient amount of carrier material which may vary from about 5 to about 95 percent of the total composition. Unit dosage forms will generally contain between from about 1 mg to about 2 g of the active ingredient, typically 25 mg, 50 mg, 100 mg, 200 mg, 300 mg, 400 mg, 500 mg, 600 mg, 800 mg, or 1000 mg.

Generally, dosage levels on the order of from about 0.01 mg/kg to about 150 mg/kg of body weight per day are useful in the treatment of the above indicated conditions, or alternatively about 0.5 mg to about 7 g per patient per day. For example, dermatological diseases and cancers may be effectively treated by the administration of from about 0.01 to 50 mg of the compound per kilogram of body weight per day, or alternatively about 0.5 mg to about 3.5 g per patient per day. It is understood, however, that the specific dose level for any particular patient will depend upon a variety of factors including the age, body weight, general health, sex, diet, time of administration, route of administration, rate of excretion, drug combination and the severity of the particular disease undergoing therapy.

The expression “unit dose” as used herein refers to a physically discrete unit of a formulation appropriate for a subject to be treated (e.g., for a single dose); each unit containing a predetermined quantity of an active agent selected to produce a desired therapeutic effect (it being understood that multiple doses may be required to achieve a desired or optimum effect), optionally together with a pharmaceutically acceptable carrier, which may be provided in a predetermined amount. The unit dose may be, for example, a volume of liquid (e.g., an acceptable carrier) containing a predetermined quantity of one or more therapeutic agents, a predetermined amount of one or more therapeutic agents in solid form, a sustained release formulation or drug delivery device containing a predetermined amount of one or more therapeutic agents, etc. It will be appreciated that a unit dose may contain a variety of components in addition to the therapeutic agent(s). For example, acceptable carriers (e.g., pharmaceutically acceptable carriers), diluents, stabilizers, buffers, preservatives, etc., may be included as described infra. It will be understood, however, that the total daily usage of a formulation of the present disclosure will be decided by the attending physician within the scope of sound medical judgment. The specific effective dose level for any particular subject or organism may depend upon a variety of factors including the disorder being treated and the severity of the disorder; activity of specific active compound employed; specific composition employed; age, body weight, general health, sex and diet of the subject; time of administration, and rate of excretion of the specific active compound employed; duration of the treatment; drugs and/or additional therapies used in combination or coincidental with specific compound(s) employed, and like factors well known in the medical arts.

Accordingly, in another aspect of the present disclosure, pharmaceutical compositions are provided, which comprise any one or more of the compounds described herein (or a prodrug, pharmaceutically acceptable salt or other pharmaceutically acceptable derivative thereof), and optionally comprise a pharmaceutically acceptable carrier. In certain embodiments, these compositions (including those that are combinations already, for example, a GPCR activator and a PDE4 inhibitor) optionally further comprise one or more additional therapeutic agents. Alternatively, a compound of this disclosure may be administered to a patient in need thereof in combination with the administration of one or more other therapeutic agents. For example, additional therapeutic agents for conjoint administration or inclusion in a pharmaceutical composition with a compound of this disclosure may be an approved agent to treat the same or related indication, or it may be any one of a number of agents undergoing approval in the Food and Drug Administration that ultimately obtain approval for the treatment of any disorder related to dysproliferation. It will also be appreciated that certain of the compounds of present disclosure can exist in free form for treatment, or where appropriate, as a pharmaceutically acceptable derivative thereof. According to the present disclosure, a pharmaceutically acceptable derivative includes, but is not limited to, pharmaceutically acceptable salts, esters, salts of such esters, or a pro drug or other adduct or derivative of a compound of this disclosure which upon administration to a patient in need is capable of providing, directly or indirectly, a compound as otherwise described herein, or a metabolite or residue thereof.

It is understood, however, that the specific dose level for any particular patient will depend upon a variety of factors including the age, body weight, general health, sex, diet, time of administration, route of administration, rate of excretion, drug combination and the severity of the particular disease undergoing therapy.

Any patent, patent application publication, or scientific publication, cited herein, is incorporated by reference herein in its entirety.

The following examples are presented in order to more fully illustrate some embodiments of the invention. They should, in no way be construed, however, as limiting the broad scope of the invention. One skilled in the art can readily devise many variations and modifications of the principles disclosed herein without departing from the scope of the invention.

### EXAMPLES

The agents and methods of the present description may be better understood by way of the following non-limiting experimental examples.

We hypothesized that molecules that inhibit the “chromatinized” Il12b reporter but not the “minimal” Il12b reporter would offer a greater degree of Il12b inhibition selectivity.

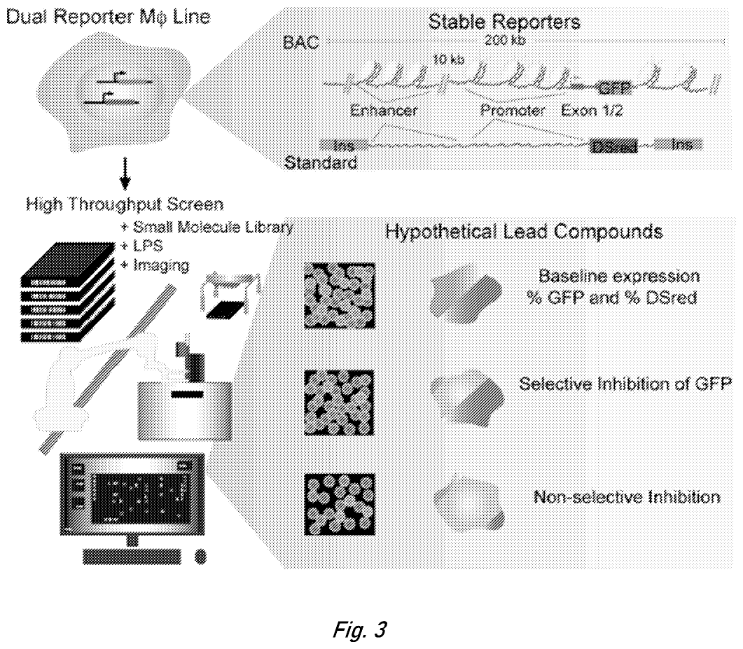

We generated a dual-fluorescence reporter macrophage cell line having both a [Il12b]-[eGFP] chromatin-based construct and a [Il12b]-[DSred] plasmid vector. FIG. 3. We then stimulated an autoimmune response in the cell line using lipopolysaccharide, an activator of Il12b expression, and tested a library of over 60,000 known and unknown (i.e., undescribed in the literature) small-molecule compounds.

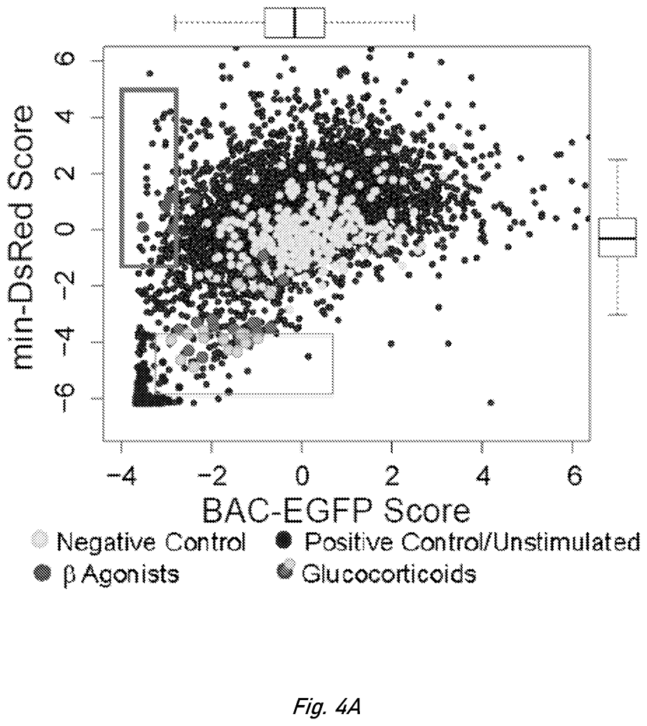

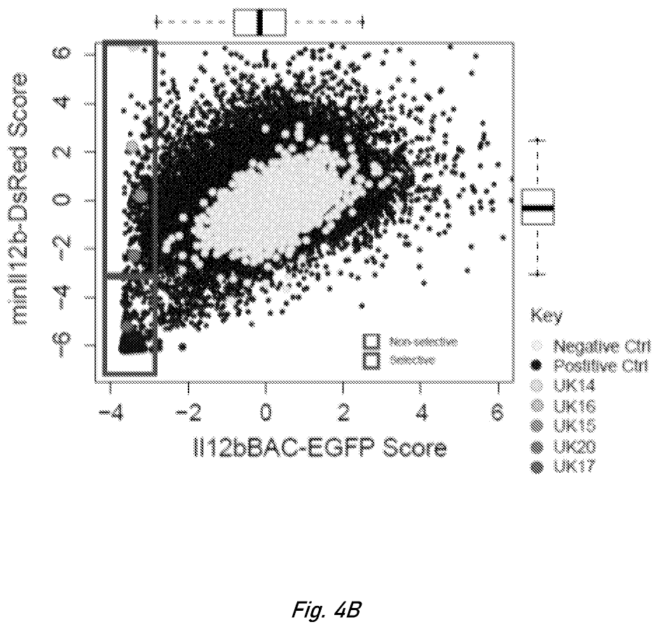

We identified several inhibitors of Il12b that fell into two main groups: (1) compounds that only inhibited detection of the chromatinized reporter; and (2) compounds that inhibited detection of both the chromatinized reporter and the minimal reporter. FIGS. 4A, 4B.

Compounds that only inhibited the chromatinized reporter included a group of compounds that activate β₂ adrenergic reception, which is a Gα protein coupled receptor (GPCR) that activates cyclic AMP (cAMP) and protein kinase A (PKA) signaling.

Our screening experiment identified as compounds that inhibited both reporters. Using ATAC-Seq and RNA-Seq analyses, we confirmed that the screen indeed identified hits that work by affecting chromatin, and confirmed that β adrenergic agonists, (and in particular β₂ adrenergic agonists) are indeed exquisitely selective inhibitors of Il12b gene expression. A poorly degradable and cell permeable form of cyclic AMP (dibutyryl cyclic AMP) and another activator of GPCRs, prostaglandin E2 (activator of the eicosanoid prostanoid receptor 2 and 4), also selectively inhibit IL12b expression.

The inhibition of Il12b by β agonists occurs upon stimulation of macrophages with other inflammatory stimuli, including CpG DNA, double stranded RNA, and imiquimod. We confirmed that the cAMP/PKA pathway, but not the exchange factor directly activated by cAMP (EPAC) pathway, was largely responsible for mediating the Il12b suppression by 0-agonists. We confirmed that prostaglandin E2 is an exquisitely selective inhibitor of Il12b in human macrophages.

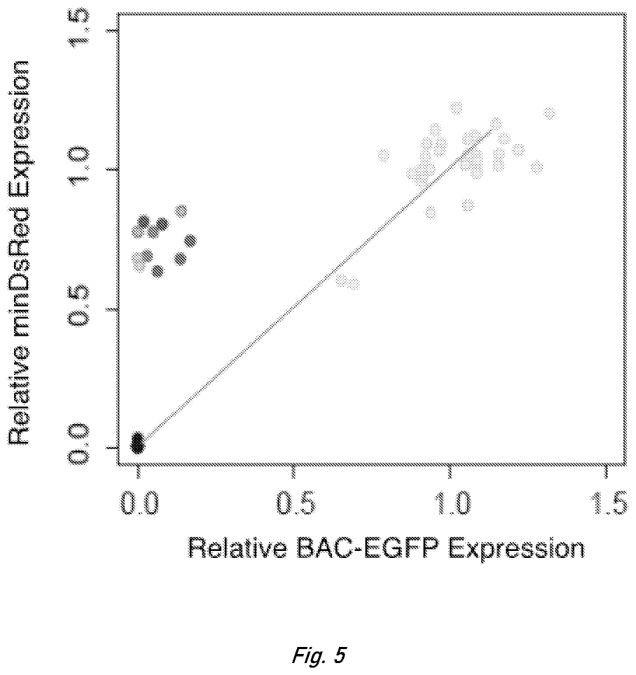

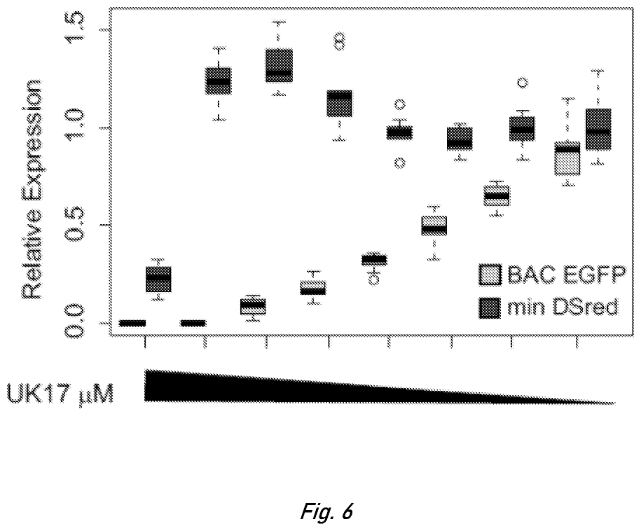

FIG. 5 depicts the relative expression levels of minDsRed and BAC-eGFP in samples treated with UK14, UK15, UK16, UK17, and UK20, where these designations have the same meaning as in FIG. 4B. FIG. 6 depicts relative expression of minDsRed constructs and BAC-eGFP constructs after treatment with a fourfold serial dilution of UK17 (3-fluoro-N′-[(5-nitro-2-thienyl)methylene]benzohydrazide (CID #6895129)), a novel compound selective for inhibition of chromatinized Il12b.

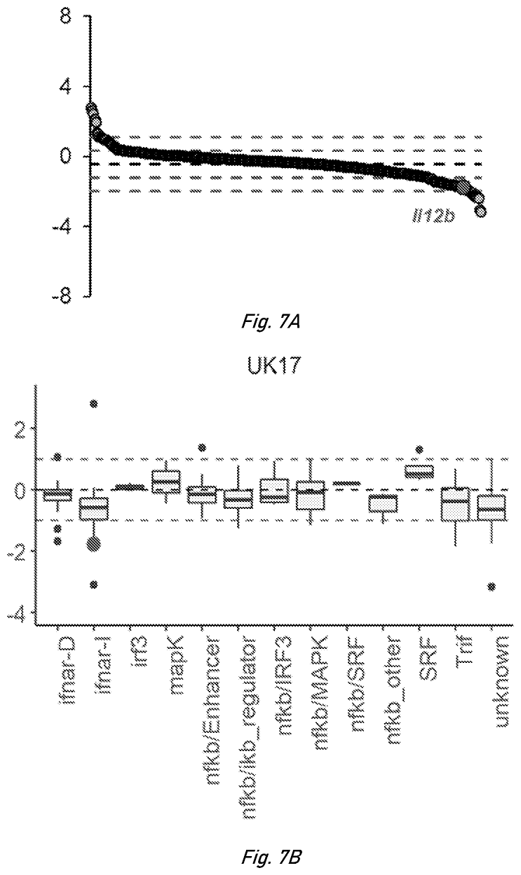

FIG. 7A depicts the effects of UK17 (3-fluoro-N′-[(5-nitro-2-thienyl)methylene]benzohydrazide (CID #6895129)) on lipopolysaccharide-induced genes (RPKM>4 and FC>10) ranked in descending fold-change order (Max RPKM with UK17/Max RPKM without UK17. Il12b ranks in the top 20 most strongly inhibited lipopolysaccharide-induced genes in samples treated with selective inhibitors of chromatinized Il12b. Black dotted lines indicate the mean of the fold change values for all lipopolysaccharide-induced genes. Blue dotted lines represent one standard deviation above or below the mean, and red dotted lines represent two standard deviations above or below the mean. FIG. 7B depicts a fold change box plot in samples treated with UK17 (3-fluoro-N′-[(5-nitro-2-thienyl)methylene]benzohydrazide (CID #6895129)), classifying lipopolysaccharide-induced genes by gene-regulation cluster as previously described in Tong A. et al., A Stringent Systems Approach Uncovers Gene-Specific Mechanisms Regulating Inflammation, Cell 165 165-179 (2016). Box plots indicate the distribution of fold change values for each cluster. Red lines are drawn at 1 and −1, indicating a 2-fold up or down-regulation of genes. Il12b is labeled in red.

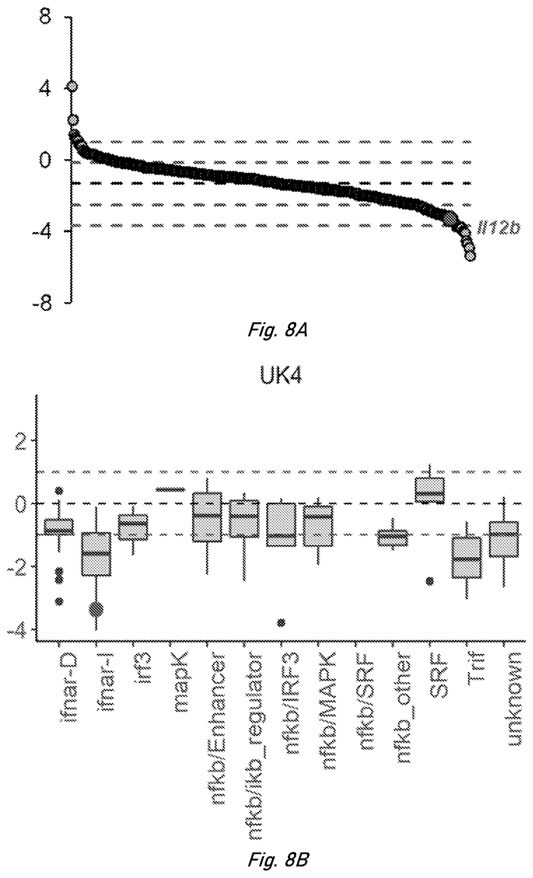

FIG. 8A depicts the effects of UK4 (3-(5-bromo-2-methoxyphenyl)-1-(3,4-dimethoxyphenyl)-2-propen-1-one (CID #5335375)) on lipopolysaccharide-induced genes (RPKM>4 and FC>10) ranked in descending fold-change order (Max RPKM with UK17/Max RPKM without UK17. Il12b ranks in the top 20 most strongly inhibited lipopolysaccharide-induced genes in samples treated with selective inhibitors of chromatinized Il12b. Black dotted lines indicate the mean of the fold change values for all lipopolysaccharide-induced genes. Blue dotted lines represent one standard deviation above or below the mean, and red dotted lines represent two standard deviations above or below the mean. FIG. 8B depicts a fold change box plot in samples treated with UK4 (3-(5-bromo-2-methoxyphenyl)-1-(3,4-dimethoxyphenyl)-2-propen-1-one (CID #5335375)), classifying lipopolysaccharide-induced genes by gene-regulation cluster as previously described in Tong et al. (2016). Box plots indicate the distribution of fold change values for each cluster. Red lines are drawn at 1 and −1, indicating a 2-fold up or down-regulation of genes. Il12b is labeled in red.

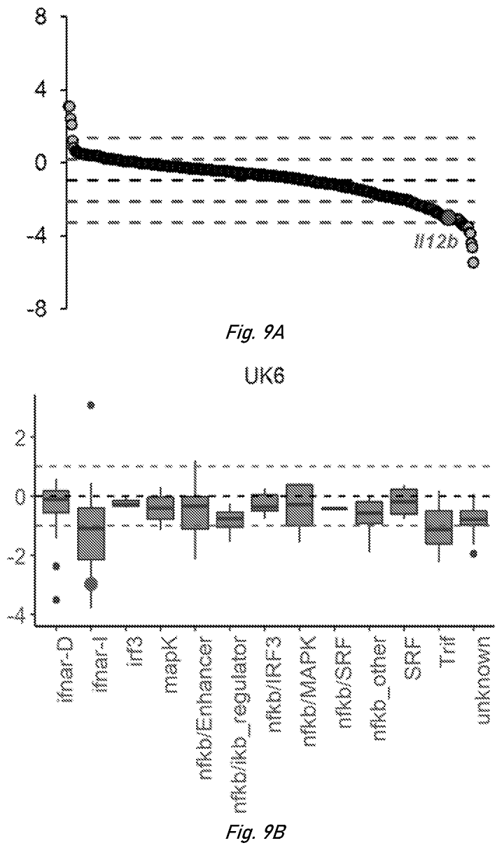

FIG. 9A depicts the effects of UK6 (5,6-dimethyl-N-[(5-nitro-2-furyl)methylene]-1H-benzimidazol-1-amine (CID #6896342)) on lipopolysaccharide-induced genes (RPKM>4 and FC>10) ranked in descending fold-change order (Max RPKM with UK17/Max RPKM without UK17. Il12b ranks in the top 20 most strongly inhibited lipopolysaccharide-induced genes in samples treated with selective inhibitors of chromatinized Il12b. Black dotted lines indicate the mean of the fold change values for all lipopolysaccharide-induced genes. Blue dotted lines represent one standard deviation above or below the mean, and red dotted lines represent two standard deviations above or below the mean. FIG. 9B depicts a fold change box plot in samples treated with UK6 (5,6-dimethyl-N-[(5-nitro-2-furyl)methylene]-1H-benzimidazol-1-amine (CID #6896342)), classifying lipopolysaccharide-induced genes by gene-regulation cluster as previously described in Tong et al. (2016). Box plots indicate the distribution of fold change values for each cluster. Red lines are drawn at 1 and −1, indicating a 2-fold up or down-regulation of genes. Il12b is labeled in red.

Among the selective inhibitors of chromatinized Il12b were: 1-(2-nitro-1-propen-1-yl)naphthalene; 2-{[(5-nitro-2-thienyl)methylene]amino}benzamide; 1-ethoxy-4-(2-nitrovinyl)benzene; 3-(5-bromo-2-methoxyphenyl)-1-(3,4-dimethoxyphenyl)-2-propen-1-one; 2-(1H-benzimidazol-2-yl)-1-phenyl-3-(3-pyridinyl)-2-propen-1-one; 5,6-dimethyl-N-[(5-nitro-2-furyl)methylene]-1H-benzimidazol-1-amine; 11-(5-hydroxy-2-nitrobenzyl)-7,11-diazatricyclo[7.3.1.0˜2,7˜]trideca-2,4-dien-6-one; 6-chloro-2-trichloromethyl)-4H-chromen-4-one; N-(5-bromo-2-biphenylyl)acetamide; diethyl [(1,3-dioxo-2,3-dihydro-1H-inden-5-yl)methylene]malonate; 5-ethylphenazin-5-ium ethyl sulfate; and 3-fluoro-N′-[(5-nitro-2-thienyl)methylene]benzohydrazide.

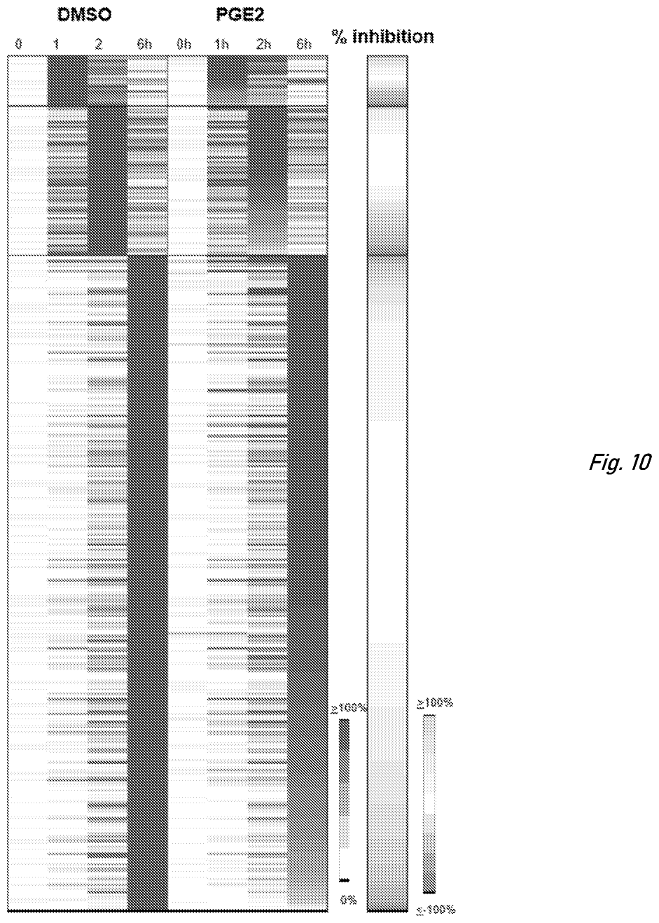

FIG. 10 depicts a heatmap of percent expression of RPKMs as a percent of the maximum RPKM within the DMSO sample for genes with RPKMs>3 and FC>10 in at least one DMSO timepoint. The heatmap represents that for the averaged samples for each gene between Donor 0 IL34 and Donor 0 MCSF. In the left heatmap, red indicates percent expression equal to or higher than 100%. DMSO and PGE2 samples are indicated as such for timepoints 0 hr, 1 hr, 2 hr, and 6 hr. The heatmap on the right shows represents percent inhibition as the difference between PGE2 samples and the DMSO samples. Here, red represents a positive difference equal to or over 100%, indicating induction, and blue representing a negative difference equal to or less than −100%, indicating inhibition. (n=2).

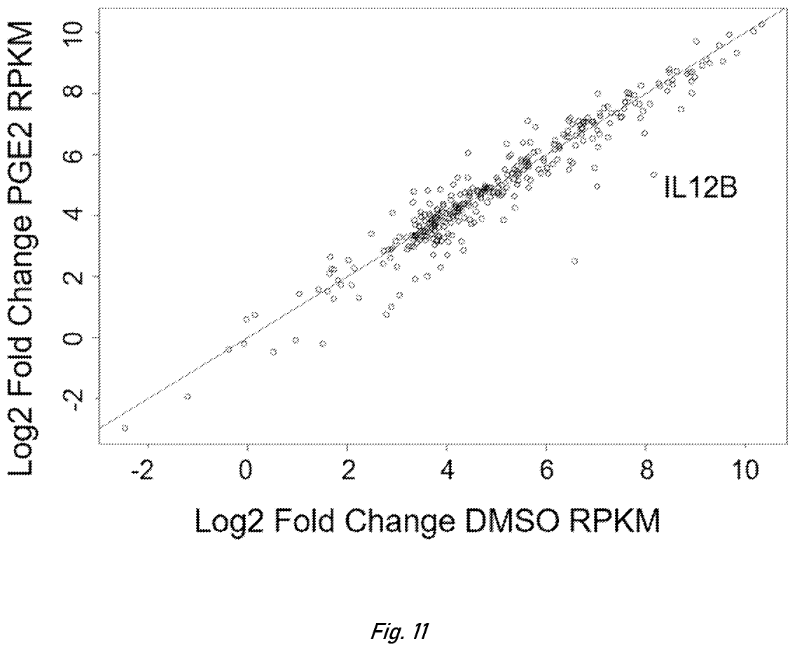

FIG. 11 depicts log 2 of the fold change of RPKMS after PGE2-treatment and those after DMSO treatment are shown in this scatter plot for genes with RPKMs>3 and FC>10 in at least on DMSO timepoint and this graph represents the 6-hour timepoint. A red line is graphed at x=y, where the RPKMs after PGE2-treatment would equal the RPKMs after DMSO treatment and a point near or on this line would indicate similarity between the two numbers. The plot point representing Il12b is indicated as such. (n=2).

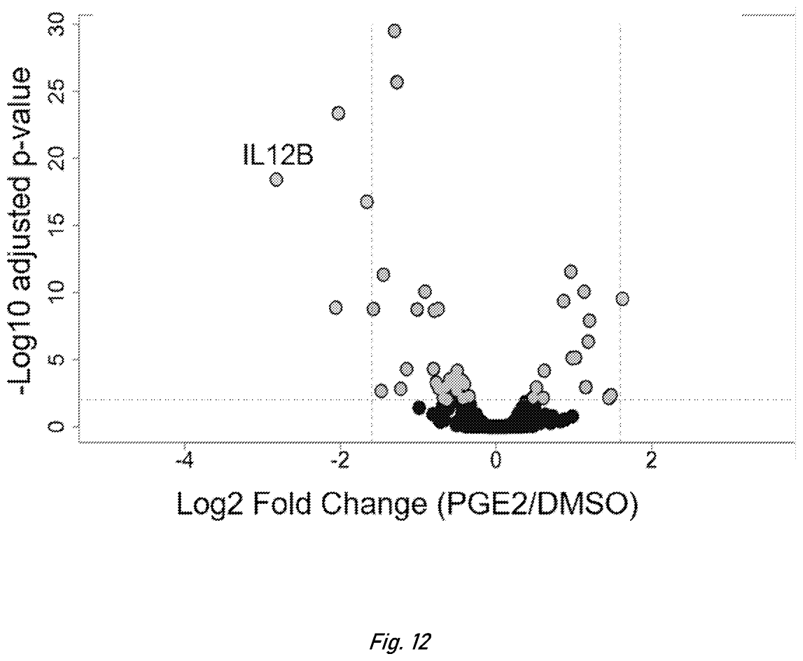

FIG. 12 depicts a volcano plot representing the −log 10 of the p-value on the y-axis and the log 2 of the fold change from PGE2-treated samples over DMSO-treated samples at the averaged 6-hour timepoint between Donor 0 IL34 and Donor 0 MCSF. Criteria for genes is set at RPKMs over 3 and fold change over 10. Il12b is indicated as such. (n=2)

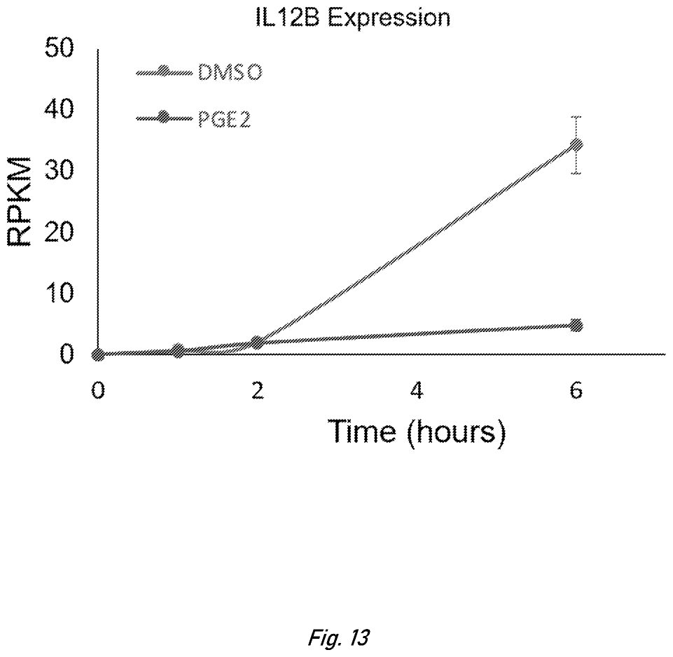

FIG. 13 depicts a graph representing the RPKMs for Il12b averaged between the two samples from Donor 0. RPKMs after DMSO treatment are graphed in blue, while RPKMs after PGE2 treatment are graphed in red. Error bars are shown in black. (n=2)

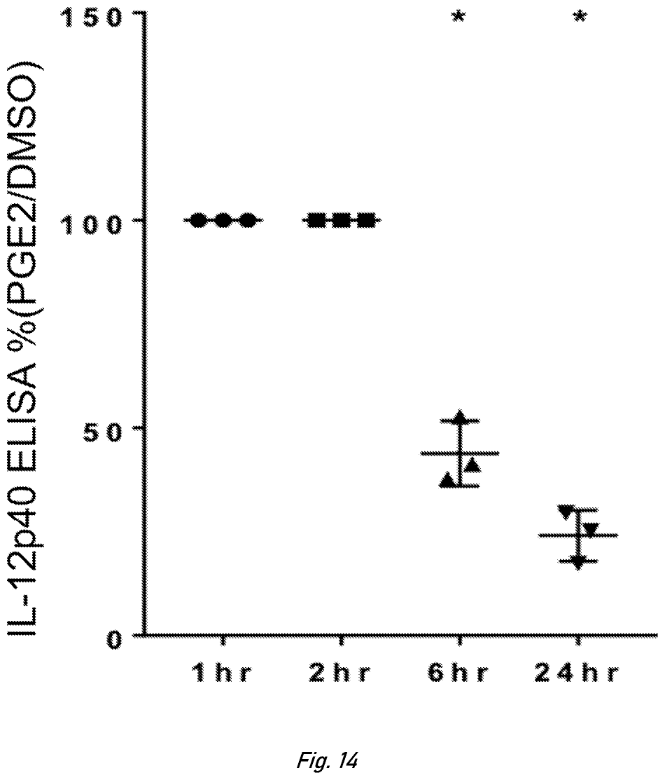

FIG. 14 depicts a graph indicating ELISA data for IL12p40 (the protein encoded by Il12b) at 1-hour, 2-hour, 6-hour, and 24-hour timepoints.

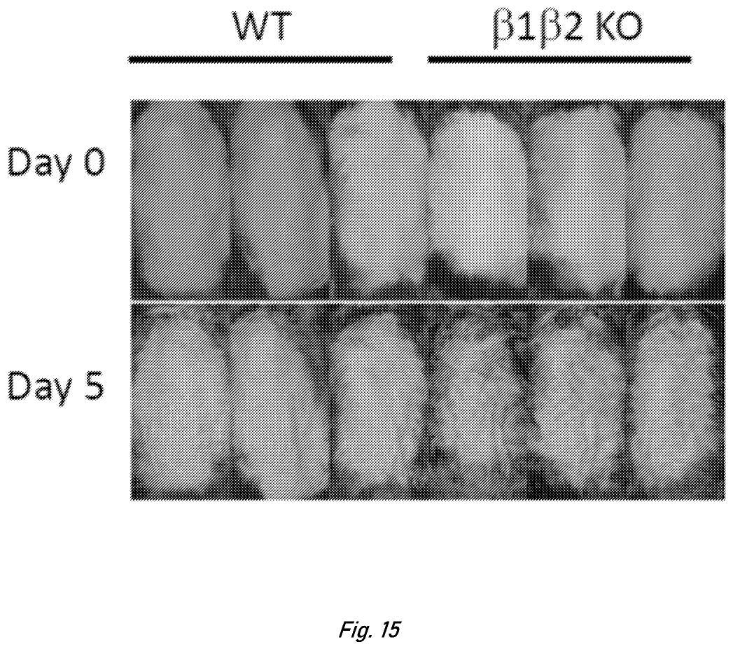

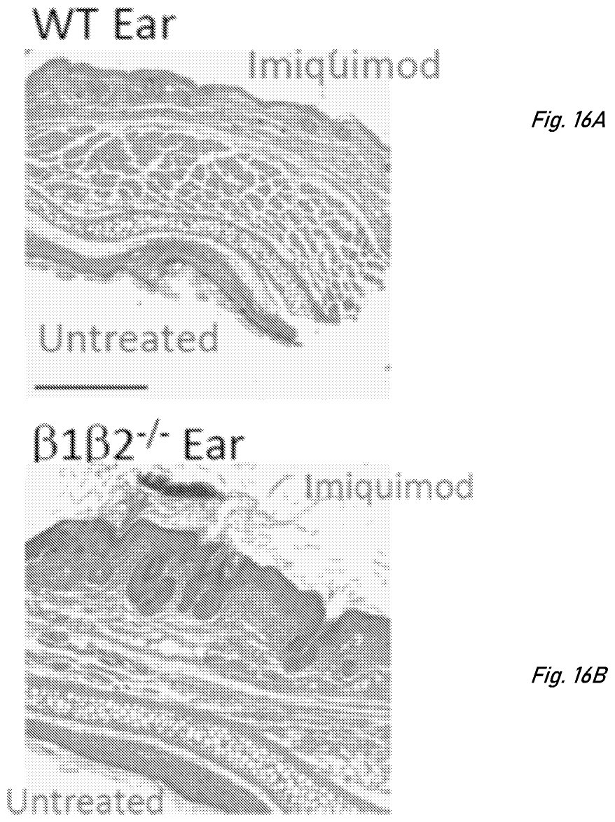

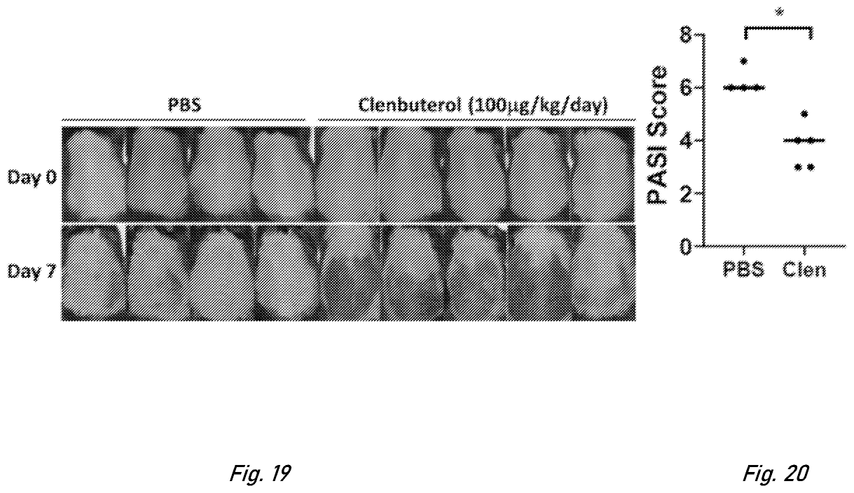

Finally, we found that genetic ablation of 31 adrenergic receptors and β2 adrenergic receptors results in worsening of skin disease in imiquimod-induced psoriasiform dermatitis model (FIGS. 15-18), confirming a regulatory role of adrenergic/sympathetic signaling in T_{H}1/T_{H}17-mediated inflammatory response, and reduction in score by systemic administration of clenbuterol (FIGS. 19, 20). FIG. 15 depicts mouse skin of wild type (WT) mice or mice lacking b1 and b2 adrenergic receptors (b1/b2 KO) 5 days after topically applying imiquimod to their skin. b1/b2 KO mice demonstrate a higher psoriasis activity and severity index (PASI) score than WT mice. FIG. 16 depicts histology of WT mice or b1/b2 KO mice 5 days after topically applying imiquimod to their skin. Histology confirms greater thickening and scale of the skin in b1/b2 KO mice.

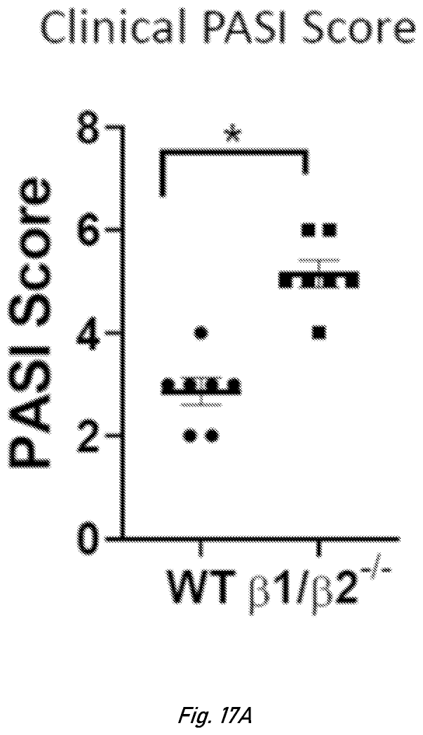

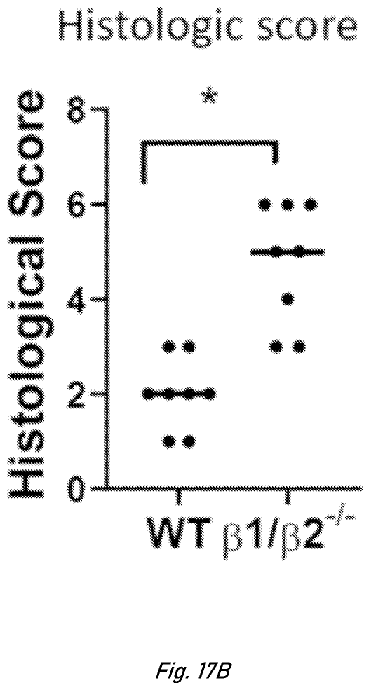

FIG. 17A depicts psoriasis area and severity index (PASI) clinical scoring of WT mice versus b1/b2 KO mice 5 days after topically applying imiquimod to their skin. FIG. 17B depicts histological scoring of WT mice versus b1/b2 KO mice 5 days after topically applying imiquimod to their skin.

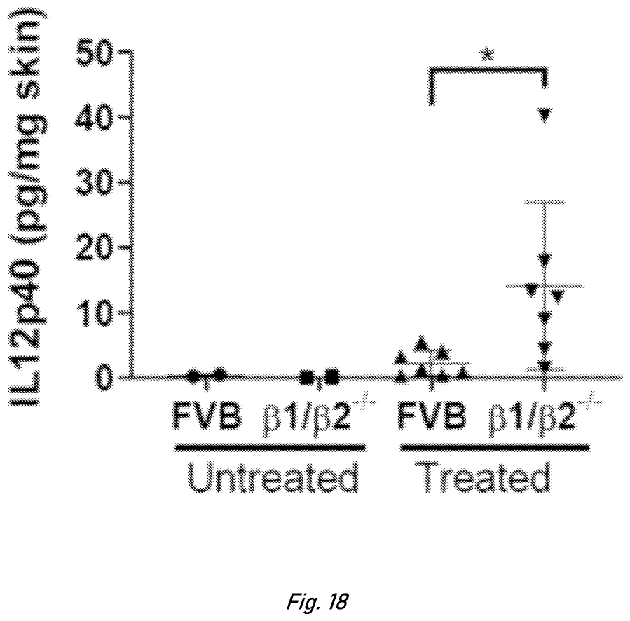

FIG. 18 depicts expression levels of IL12p40 (picograms per milligram of skin) in FVB strain mice versus b1/b2 KO mice untreated and treated with b2 selective adrenergic agonist.

FIG. 19 depicts WT mice given intraperitoneal injection of the b2 selective adrenergic agonist clenbuterol or vehicle control (saline) daily in mice given topical imiquimod to induce psoriasiform dermatitis.

FIG. 20 depicts the PASI clinical scoring PBS-treated WT mice versus clenbuterol-treated WT mice.

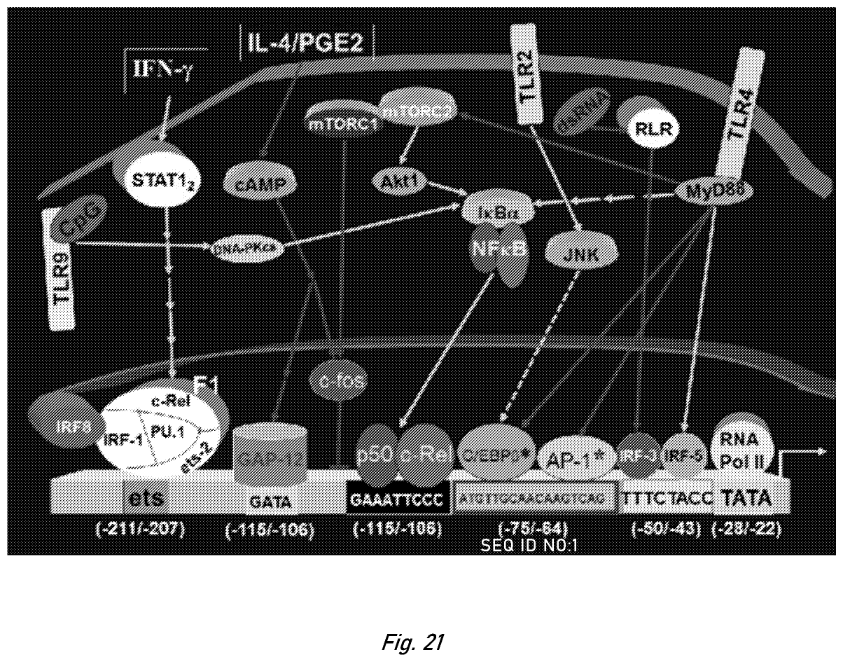

FIG. 21 depicts a schematic cartoon of Il12b regulatory pathway as had been understood prior to the present disclosure.

Additional compounds in Formulas (I)-(IV) were screened following the same procedure as described above, with eGFP fluorescence as a readout for inhibition of IL12b. Positive activity was identified in the following compounds: I-2: N-[(5,8-dimethyl-2-oxo-1H-quinolin-3-yl)methyl]-N-[(4-fluorophenyl)methyl]-3,5-dimethoxybenzamide; I-3: 4-tert-butyl-N-[(6-ethoxy-2-oxo-1H-quinolin-3-yl)methyl]-N-(furan-2-ylmethyl)benzamide; I-4: 1-(furan-2-ylmethyl)-3-(4-methylphenyl)-1-[(7-oxo-3,6-dihydro-2H-[1,4]dioxino[2,3-g]quinolin-8-yl)methyl]urea; I-5:
1-[(6-ethoxy-2-oxo-1H-quinolin-3-yl)methyl]-1-(furan-2-ylmethyl)-3-(4-methoxyphenyl)urea; I-6: 3-(4-ethoxyphenyl)-1-(furan-2-ylmethyl)-1-[(7-methyl-2-oxo-1H-quinolin-3-yl)methyl]thiourea; I-7: 1-(furan-2-ylmethyl)-3-(4-methylphenyl)-1-[(6-oxo-5H-[1,3]dioxolo[4,5-g]quinolin-7-yl)methyl]urea; I-8: 3-benzyl-1-(furan-2-ylmethyl)-1-[(6-oxo-5H-[1,3]dioxolo[4,5-g]quinolin-7-yl)methyl]urea; I-9: 3-benzyl-1-[(6-ethyl-2-oxo-1H-quinolin-3-yl)methyl]-1-(furan-2-ylmethyl)thiourea; I-10: 1-[(5,8-dimethyl-2-oxo-1H-quinolin-3-yl)methyl]-1,3-bis(furan-2-ylmethyl)thiourea; I-11: N-[(6-ethoxy-2-oxo-1H-quinolin-3-yl)methyl]-N-(furan-2-ylmethyl)thiophene-2-carboxamide; II-2: N—[(Z)-(5-nitrothiophen-2-yl)methylideneamino]-3-phenylpropanamide; II-3: N—[(Z)-(5-nitrofuran-2-yl)methylideneamino]-4-phenylbenzamide; II-4: 3-chloro-N-[(E)-1-(5-nitrofuran-2-yl)ethylideneamino]benzamide; II-5: 2-(4-chlorophenyl)-N—[(Z)-(5-nitrofuran-2-yl)methylideneamino]acetamide; II-6: N—[(Z)-(5-nitrofuran-2-yl)methylideneamino]naphthalene-1-carboxamide; III-2 (1R,9S)-11-(pyren-1-ylmethyl)-7,11-diazatricyclo[7.3.1.0^{2,7}]trideca-2,4-dien-6-one; IV-2 (E)-3-(5-bromo-2-methoxyphenyl)-1-(2,3-dihydro-1,4-benzodioxin-6-yl)prop-2-en-1-one; IV-3: (E)-3-(2,5-dimethoxyphenyl)-1-(4-methylphenyl)prop-2-en-1-one; IV-4: €-3-(3-chlorophenyl)-1-(3,4-dimethoxyphenyl)prop-2-en-1-one; IV-5: (E)-3-(2,3-dimethoxyphenyl)-1-(4-ethoxyphenyl)prop-2-en-1-one.

### TREATMENT EXAMPLES

A patient with psoriasis is treated with a topical formulation of N-(2-furylmethyl)-N-[(6-methoxy-2-oxo-1H-quinolin-3-yl)methyl]-4-methyl-benzamide (Compound I-1). Resolution of symptoms is observed.

A patient with chronic eczema is treated with a topical formulation of (3-fluoro-N′-[(5-nitro-2-thienyl)methylene]benzohydrazide (Compound II-1). Resolution of symptoms is observed.

A patient with vitiligo is treated with a topical formulation of 11-(5-hydroxy-2-nitrobenzyl)-7,11-diazatricyclo[7.3.1.0˜2,7˜]trideca-2,4-dien-6-one (Compound III-1). Resolution of vitiligo is observed.

A patient with Behçet's disease is treated with an topical formulation of (3-(5-bromo-2-methoxyphenyl)-1-(3,4-dimethoxyphenyl)-2-propen-1-one (Compound IV-1). Resolution of disease is observed.

A patient with lichen planus is treated with a combination of N-(2-furylmethyl)-N-[(6-methoxy-2-oxo-1H-quinolin-3-yl)methyl]-4-methyl-benzamide (Compound I-1) and crisaborole, both administered topically. Resolution of symptoms is observed.

A patient with alopecia is treated with a topical formulation comprising (3-(5-bromo-2-methoxyphenyl)-1-(3,4-dimethoxyphenyl)-2-propen-1-one (Compound IV-1) and apremilast. Resolution of alopecia is observed.

While certain features have been illustrated and described herein, many modifications, substitutions, changes, and equivalents will now occur to those of ordinary skill in the art. It is, therefore, to be understood that the appended claims are intended to cover all such modifications and changes as fall within the true spirit of the disclosure.

What is claimed is:

1. A method of treating an inflammatory disease in a subject comprising administering a therapeutically effective dose of a selective Il12b inhibitor of Formula (I): 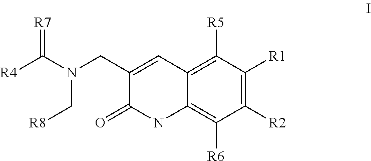
wherein R1 and R2 are independently hydrogen, alkyl, or alkyloxy; or R1 and R2 taken together is methylenedioxy or ethylenedioxy;
R8 is thienyl, furyl or 4-halophenyl;
R4 is 4-alkylphenyl, 2,4-dialkoxyphenyl, 4-alkylphenylamino, 4-alkoxyphenylamino, benzylamino, furylalkylamino or thienyl;
R5 and R6 are independently hydrogen or alkyl;
and R7 is sulfur or oxygen.

2. The method of claim 1 wherein R1 and R2 are independently hydrogen, ethoxy, methoxy or methyl; or R1 and R2 taken together is ethylenedioxy; and/or R8 is 2-thienyl, 2-furyl or 4-fluorophenyl; and/or R4 is 4-methylphenyl, 4-t-butyl-phenyl, 2,4-dimethoxyphenyl, 4-methylphenylamino, 4-ethoxyphenylamino, benzylamino, furylmethylamino or thienyl.

3. The method of claim 1 wherein the compound is N-(2-furylmethyl)-N-[(6-methoxy-2-oxo-1H-quinolin-3-yl)methyl]-4-methyl-benzamide; N-[(5,8-dimethyl-2-oxo-1H-quinolin-3-yl)methyl]-N-[(4-fluorophenyl)methyl]-3,5-dimethoxybenzamide; 4-tert-butyl-N-[(6-ethoxy-2-oxo-1H-quinolin-3-yl)methyl]-N-(furan-2-ylmethyl)benzamide; 1-(furan-2-ylmethyl)-3-(4-methylphenyl)-1-[(7-oxo-3,6-dihydro-2H-[1,4]dioxino[2,3-g]quinolin-8-yl)methyl]urea; 1-[(6-ethoxy-2-oxo-1H-quinolin-3-yl)methyl]-1-(furan-2-ylmethyl)-3-(4-methoxyphenyl)urea; 3-(4-ethoxyphenyl)-1-(furan-2-ylmethyl)-1-[(7-methyl-2-oxo-1H-quinolin-3-yl)methyl]thiourea; 1-(furan-2-ylmethyl)-3-(4-methylphenyl)-1-[(6-oxo-5H-[1,3]dioxolo[4,5-g]quinolin-7-yl)methyl]urea; 3-benzyl-1-(furan-2-ylmethyl)-1-[(6-oxo-5H-[1,3]dioxolo[4,5-g]quinolin-7-yl)methyl]urea; 3-benzyl-1-[(6-ethyl-2-oxo-1H-quinolin-3-yl)methyl]-1-(furan-2-ylmethyl)thiourea; 1-[(5,8-dimethyl-2-oxo-1H-quinolin-3-yl)methyl]-1,3-bis(furan-2-ylmethyl)thiourea; or N-[(6-ethoxy-2-oxo-1H-quinolin-3-yl)methyl]-N-(furan-2-ylmethyl)thiophene-2-carboxamide.

4. A method of treating an inflammatory disease in a subject comprising administering a therapeutically effective dose of a selective Il12b inhibitor of Formula (II): 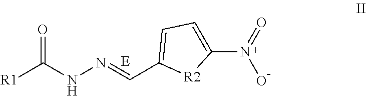
wherein R1 is aryl optionally substituted by one or more halo, alkyl or arylalkyl; and
R2 is oxygen or sulfur.

5. The method of claim 4 wherein R1 is phenyl optionally substituted with one or more halo or alkyl, such as 4-halophenyl or 3-halophenyl; and/or R1 is 4-(1,1′-biphenyl), naphthyl such as 1-naphthyl, phenylalkyl. Phenylethyl, or naphthylalkyl such as 1-naphthylmethyl.

6. The method of claim 4 wherein the compound is 3-fluoro-N′-[(5-nitro-2-thienyl)methylene]benzohydrazide; N—[(Z)-(5-nitrothiophen-2-yl)methylideneamino]-3-phenylpropanamide; II-3: N—[(Z)-(5-nitrofuran-2-yl)methylideneamino]-4-phenylbenzamide; II-4: 3-chloro-N-[(E)-1-(5-nitrofuran-2-yl)ethylideneamino]benzamide; 2-(4-chlorophenyl)-N—[(Z)-(5-nitrofuran-2-yl)methylideneamino]acetamide; and N—[(Z)-(5-nitrofuran-2-yl)methylideneamino]naphthalene-1-carboxamide.

7. A method of treating an inflammatory disease in a subject comprising administering a therapeutically effective dose of a selective Il12b inhibitor of Formula (III): 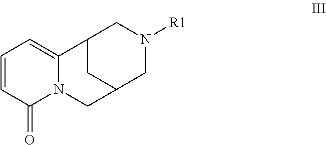
wherein R1 is benzyl optionally substituted with one or more halo, alkoxy, hydroxy, nitro, alkylthio, ethylenedioxy, aryl or benzyloxy groups, or R1 is a 1,3a-dihydropyrenylmethyl.

8. The method of claim 7 wherein R1 is benzyl substituted with one or more halo, alkoxy or hydroxy groups, such as a halo and an alkoxy group.

9. The method of claim 7 wherein the compound is 11-(5-hydroxy-2-nitrobenzyl)-7,11-diazatricyclo[7.3.1.0˜2,7˜]trideca-2,4-dien-6-one or (1R,9S)-11-(pyren-1-ylmethyl)-7,11-diazatricyclo[7.3.1.0^{2,7}]trideca-2,4-dien-6-one.

10. A method of treating an inflammatory disease in a subject comprising administering a therapeutically effective dose of a selective Il12b inhibitor of Formula (IV): 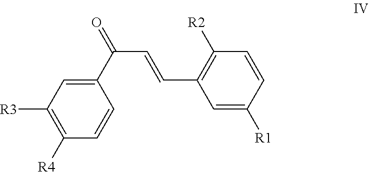
wherein R1 is halo or alkyloxy;
R2 and R3 are independently alkyloxy; and
R4 is hydrogen, alkyl or alkoxy.

11. The method of claim 10 wherein R1 is bromo or methoxy; and/or R2 is methoxy or ethoxy; and/or R3 is methoxy; and/or R4 is methyl or methoxy.

12. The method of claim 10 wherein the compound is 3-(5-bromo-2-methoxyphenyl)-1-(3,4-dimethoxyphenyl)-2-propen-1-one; (E)-3-(5-bromo-2-methoxyphenyl)-1-(2,3-dihydro-1,4-benzodioxin-6-yl)prop-2-en-1-one; (E)-3-(2,5-dimethoxyphenyl)-1-(4-methylphenyl)prop-2-en-1-one; (E)-3-(3-chlorophenyl)-1-(3,4-dimethoxyphenyl)prop-2-en-1-one; and (E)-3-(2,3-dimethoxyphenyl)-1-(4-ethoxyphenyl)prop-2-en-1-one.

13. A method of treating an inflammatory disease in a subject comprising administering a therapeutically effective dose of a selective Il12b inhibitor, wherein the selective Il12b inhibitor is selected from 1-(2-nitro-1-propen-1-yl)naphthalene; 2-{[(5-nitro-2-thienyl)methylene]amino}benzamide; 1-ethoxy-4-(2-nitrovinyl)benzene; 3-(5-bromo-2-methoxyphenyl)-1-(3,4-dimethoxyphenyl)-2-propen-1-one; 2-(1H-benzimidazol-2-yl)-1-phenyl-3-(3-pyridinyl)-2-propen-1-one; 5,6-dimethyl-N-[(5-nitro-2-furyl)methylene]-1H-benzimidazol-1-amine; 11-(5-hydroxy-2-nitrobenzyl)-7,11-diazatricyclo[7.3.1.0˜2,7˜]trideca-2,4-dien-6-one; 6-chloro-2-trichloromethyl)-4H-chromen-4-one; N-(5-bromo-2-biphenylyl)acetamide; diethyl [(1,3-dioxo-2,3-dihydro-1H-inden-5-yl)methylene]malonate; 5-ethylphenazin-5-ium ethyl sulfate; and 3-fluoro-N′-[(5-nitro-2-thienyl)methylene]benzohydrazide, or an analogue of any of the foregoing.

14. A method of treating an inflammatory disease in a subject comprising administering a therapeutically effective dose of a selective Il12b inhibitor, wherein the selective Il12b inhibitor is selected from: 1-(2-nitro-1-propen-1-yl)naphthalene; 2-{[(5-nitro-2-thienyl)methylene]amino}benzamide; 1-ethoxy-4-(2-nitrovinyl)benzene; 3-(5-bromo-2-methoxyphenyl)-1-(3,4-dimethoxyphenyl)-2-propen-1-one; 2-(1H-benzimidazol-2-yl)-1-phenyl-3-(3-pyridinyl)-2-propen-1-one; 5,6-dimethyl-N-[(5-nitro-2-furyl)methylene]-1H-benzimidazol-1-amine; 11-(5-hydroxy-2-nitrobenzyl)-7,11-diazatricyclo[7.3.1.0˜2,7˜]trideca-2,4-dien-6-one; 6-chloro-2-(trichloromethyl)-4H-chromen-4-one; N-(5-bromo-2-biphenylyl)acetamide; diethyl [(1,3-dioxo-2,3-dihydro-1H-inden-5-yl)methylene]malonate; 5-ethylphenazin-5-ium ethyl sulfate; 1,2-dimethoxy-4-[(E)-2-nitroethenyl]benzene; 4-{[(1E,2E)-3-(5-nitrofuran-2-yl)prop-2-en-1-ylidene]amino}-5-phenyl-4H-1,2,4-triazole-3-thiol; (E)-1-ethoxy-2-(2-nitroprop-1-en-1-yl)benzene; (5E)-1-(3,4-Dimethylphenyl)-5-{[1-(4-methoxyphenyl)-1H-pyrrol-2-yl]methylidene}-2-thioxodihydropyrimidine-4,6(1H,5H)-dione; (1E,4E)-1,5-Bis(2-methoxyphenyl)penta-1,4-dien-3-one; 3-Fluoro-N′-[(E)-(5-nitrothiophen-2-yl)methylidene]benzohydrazide; 3-Methyl-5-(6-nitro-benzo[1,3]dioxol-5-ylmethylene)-2-thioxo-thiazolidin-4-one; 1-(2,3-Dihydro-benzo[1,4]dioxin-6-yl)-3-(3,4,5-trimethoxy-phenyl)-propenone; N-(2-Furylmethyl)-N-[(6-methoxy-2-oxo-1H-quinolin-3-yl)methyl]-4-methyl-benzamide; and N-[2-Chloro-5-(trifluoromethyl)phenyl]-5-nitrofuran-2-carboxamide.

15. A method of treating an inflammatory disease in a live animal comprising administering a therapeutically effective dose of a selective Il12b inhibitor, wherein the selective Il12b inhibitor is a Gα GPCR activator of cAMP signaling.

16. A method of treating an inflammatory disease in a live animal comprising administering a therapeutically effective dose of a selective Il12b inhibitor, wherein the selective Il12b inhibitor is a Gαs-subtype GPCR activator.

17. A method of treating an inflammatory disease in a live animal comprising administering a therapeutically effective dose of a selective Il12b inhibitor, wherein the selective Il12b inhibitor is a Gαi/o-subtype GPCR activator.

18. A method of treating an inflammatory disease in a live animal comprising administering a therapeutically effective dose of a selective Il12b inhibitor, wherein the selective Il12b inhibitor is a Gαq/11-subtype GPCR activator.

19. A method of treating an inflammatory disease in a live animal comprising administering a therapeutically effective dose of a selective Il12b inhibitor, wherein the selective Il12b inhibitor is a Gαl2/13-subtype GPCR activator.

20. A method of treating an inflammatory disease in a live animal comprising administering a therapeutically effective dose of a selective Il12b inhibitor, wherein the selective Il12b inhibitor is a β adrenergic activator.

21. The method of claim 20 wherein the β adrenergic activator is a β adrenergic agonist.

22. The method of claim 21 wherein the β adrenergic agonist is denopamine, dobutamine, dopexamine, epinephrine, isoprenaline, isoproterenol, prenalterol, xamoterol, arformoterol, buphenine, clenbuterol, fenoterol, formoterol, isoetarine, levosalbutamol, levalbuterol, orciprenaline, metaproterenol, pirbuterol, procaterol, ritodrine, salbutamol, albuterol, salmeterol or terbutaline.

23. A method of treating an inflammatory disease in a live animal comprising administering a therapeutically effective dose of a selective Il12b inhibitor, wherein the selective Il12b inhibitor is a β2 adrenergic activator.

24. The method of claim 23 wherein the β2 adrenergic agonist is arformoterol, buphenine, clenbuterol, dopexamine, epinephrine, fenoterol, formoterol, isoetarine. isoprenaline, isoproterenol, levosalbutamol, levalbuterol, orciprenaline, metaproterenol, pirbuterol, procaterol, ritodrine, salbutamol, albuterol, salmeterol or terbutaline.

25. A method of treating an inflammatory disease in a live animal comprising administering a therapeutically effective dose of a combination of
a. a composition comprising a Gα protein coupled receptor activator that activates cyclic AMP signaling; and
b. a composition comprising a selective inhibitor of cyclic AMP breakdown.

26. The method of claim 25 wherein the Gα protein coupled receptor activator that activates cyclic AMP signaling is a Gα GPCR activator, Gαs-subtype GPCR activator, Gαi/o-subtype GPCR activator, Gαq/11-subtype GPCR activator, Gαl2/13-subtype GPCR activator, β adrenergic activator, β adrenergic agonist, β2 adrenergic activator or a β2 adrenergic agonist.

27. The method of claim 26 wherein the β adrenergic agonist is denopamine, dobutamine, dopexamine, epinephrine, isoprenaline, isoproterenol, prenalterol, xamoterol, arformoterol, buphenine, clenbuterol, fenoterol, formoterol, isoetarine, levosalbutamol, levalbuterol, orciprenaline, metaproterenol, pirbuterol, procaterol, ritodrine, salbutamol, albuterol, salmeterol or terbutaline.

28. The method of claim 26 wherein the β2 adrenergic agonist is arformoterol, buphenine, clenbuterol, dopexamine, epinephrine, fenoterol, formoterol, isoetarine. isoprenaline, isoproterenol, levosalbutamol, levalbuterol, orciprenaline, metaproterenol, pirbuterol, procaterol, ritodrine, salbutamol, albuterol, salmeterol or terbutaline.

29. The method of claim 25 wherein the inhibitor of cyclic AMP breakdown is selected from the group consisting of mesembrenone, rolipram, ibudilast, piclimilast, luteolin, drotaverine, roflumilast, crisaborole, and apremilast.

30. The method of claim 25 wherein each one or both compositions are administered topically in the form of a cream, salve, gel, dermal patch, aqueous solution, or non-aqueous solution.

31. The method of any one of claims 1-28 wherein the inflammatory disease is any of psoriasis, chronic eczema, vitiligo, lichen planus, cutaneous lupus erythematosus, Behçet's disease, ulcerative colitis, Crohn's disease, or alopecia.

32. The method of any one of claims 15-31 wherein the inhibitor is 1-(2-nitro-1-propen-1-yl)naphthalene; 2-{[(5-nitro-2-thienyl)methylene]amino}benzamide; 1-ethoxy-4-(2-nitrovinyl)benzene; 3-(5-bromo-2-methoxyphenyl)-1-(3,4-dimethoxyphenyl)-2-propen-1-one; 2-(1H-benzimidazol-2-yl)-1-phenyl-3-(3-pyridinyl)-2-propen-1-one; 5,6-dimethyl-N-[(5-nitro-2-furyl)methylene]-1H-benzimidazol-1-amine; 11-(5-hydroxy-2-nitrobenzyl)-7,11-diazatricyclo[7.3.1.0˜2,7˜]trideca-2,4-dien-6-one; 6-chloro-2-trichloromethyl)-4H-chromen-4-one; N-(5-bromo-2-biphenylyl)acetamide; diethyl [(1,3-dioxo-2,3-dihydro-1H-inden-5-yl)methylene]malonate; 5-ethylphenazin-5-ium ethyl sulfate; or 3-fluoro-N′-[(5-nitro-2-thienyl)methylene]benzohydrazide.

33. A method of treating an inflammatory disease in a live animal comprising administering a therapeutically effective dose of a combination of
a. a compound of any one of Formulas (I)-(IV) or 1-(2-nitro-1-propen-1-yl)naphthalene; 2-{[(5-nitro-2-thienyl)methylene]amino}benzamide; 1-ethoxy-4-(2-nitrovinyl)benzene; 3-(5-bromo-2-methoxyphenyl)-1-(3,4-dimethoxyphenyl)-2-propen-1-one; 2-(1H-benzimidazol-2-yl)-1-phenyl-3-(3-pyridinyl)-2-propen-1-one; 5,6-dimethyl-N-[(5-nitro-2-furyl)methylene]-1H-benzimidazol-1-amine; 11-(5-hydroxy-2-nitrobenzyl)-7,11-diazatricyclo[7.3.1.0˜2,7˜]trideca-2,4-dien-6-one; 6-chloro-2-(trichloromethyl)-4H-chromen-4-one; N-(5-bromo-2-biphenylyl)acetamide; diethyl [(1,3-dioxo-2,3-dihydro-1H-inden-5-yl)methylene]malonate; 5-ethylphenazin-5-ium ethyl sulfate; 1,2-dimethoxy-4-[(E)-2-nitroethenyl]benzene; 4-{[(1E,2E)-3-(5-nitrofuran-2-yl)prop-2-en-1-ylidene]amino}-5-phenyl-4H-1,2,4-triazole-3-thiol; (E)-1-ethoxy-2-(2-nitroprop-1-en-1-yl)benzene; (5E)-1-(3,4-Dimethylphenyl)-5-{[1-(4-methoxyphenyl)-1H-pyrrol-2-yl]methylidene}-2-thioxodihydropyrimidine-4,6(1H,5H)-dione; (1E,4E)-1,5-Bis(2-methoxyphenyl)penta-1,4-dien-3-one; 3-Fluoro-N′-[(E)-(5-nitrothiophen-2-yl)methylidene]benzohydrazide; 3-Methyl-5-(6-nitro-benzo[1,3]dioxol-5-ylmethylene)-2-thioxo-thiazolidin-4-one; 1-(2,3-Dihydro-benzo[1,4]dioxin-6-yl)-3-(3,4,5-trimethoxy-phenyl)-propenone; N-(2-Furylmethyl)-N-[(6-methoxy-2-oxo-1H-quinolin-3-yl)methyl]-4-methyl-benzamide; and N-[2-Chloro-5-(trifluoromethyl)phenyl]-5-nitrofuran-2-carboxamide; and
b. a composition comprising a selective inhibitor of cyclic AMP breakdown.

34. The method of claim 33 wherein the compound of any one of Formula (I)-(IV) is N-[(5,8-dimethyl-2-oxo-1H-quinolin-3-yl)methyl]-N-[(4-fluorophenyl)methyl]-3,5-dimethoxybenzamide; 4-tert-butyl-N-[(6-ethoxy-2-oxo-1H-quinolin-3-yl)methyl]-N-(furan-2-ylmethyl)benzamide; 1-(furan-2-ylmethyl)-3-(4-methylphenyl)-1-[(7-oxo-3,6-dihydro-2H-[1,4]dioxino[2,3-g]quinolin-8-yl)methyl]urea; 1-[(6-ethoxy-2-oxo-1H-quinolin-3-yl)methyl]-1-(furan-2-ylmethyl)-3-(4-methoxyphenyl)urea; 3-(4-ethoxyphenyl)-1-(furan-2-ylmethyl)-1-[(7-methyl-2-oxo-1H-quinolin-3-yl)methyl]thiourea; 1-(furan-2-ylmethyl)-3-(4-methylphenyl)-1-[(6-oxo-5H-[1,3]dioxolo[4,5-g]quinolin-7-yl)methyl]urea; 3-benzyl-1-(furan-2-ylmethyl)-1-[(6-oxo-5H-[1,3]dioxolo[4,5-g]quinolin-7-yl)methyl]urea; 3-benzyl-1-[(6-ethyl-2-oxo-1H-quinolin-3-yl)methyl]-1-(furan-2-ylmethyl)thiourea; 1-[(5,8-dimethyl-2-oxo-1H-quinolin-3-yl)methyl]-1,3-bis(furan-2-ylmethyl)thiourea; N-[(6-ethoxy-2-oxo-1H-quinolin-3-yl)methyl]-N-(furan-2-ylmethyl)thiophene-2-carboxamide; N—[(Z)-(5-nitrothiophen-2-yl)methylideneamino]-3-phenylpropanamide; N—[(Z)-(5-nitrofuran-2-yl)methylideneamino]-4-phenylbenzamide; 3-chloro-N-[(E)-1-(5-nitrofuran-2-yl)ethylideneamino]benzamide; 2-(4-chlorophenyl)-N—[(Z)-(5-nitrofuran-2-yl)methylideneamino]acetamide; N—[(Z)-(5-nitrofuran-2-yl)methylideneamino]naphthalene-1-carboxamide; (1R,9S)-11-(pyren-1-ylmethyl)-7,11-diazatricyclo[7.3.1.0^{2,7}]trideca-2,4-dien-6-one; (E)-3-(5-bromo-2-methoxyphenyl)-1-(2,3-dihydro-1,4-benzodioxin-6-yl)prop-2-en-1-one; (E)-3-(2,5-dimethoxyphenyl)-1-(4-methylphenyl)prop-2-en-1-one; (E)-3-(3-chlorophenyl)-1-(3,4-dimethoxyphenyl)prop-2-en-1-one; or (E)-3-(2,3-dimethoxyphenyl)-1-(4-ethoxyphenyl)prop-2-en-1-one.

35. The method of claim 33 wherein the selective inhibitor of cyclic AMP breakdown is selected from the group consisting of mesembrenone, rolipram, ibudilast, piclimilast, luteolin, drotaverine, roflumilast, crisaborole, and apremilast.

36. The method of claim 33 wherein each one or both compositions are administered topically in the form of a cream, salve, gel, dermal patch, aqueous solution, or non-aqueous solution.

37. The method of claim 33 wherein the inhibitor is 1-(2-nitro-1-propen-1-yl)naphthalene; 2-{[(5-nitro-2-thienyl)methylene]amino}benzamide; 1-ethoxy-4-(2-nitrovinyl)benzene; 3-(5-bromo-2-methoxyphenyl)-1-(3,4-dimethoxyphenyl)-2-propen-1-one; 2-(1H-benzimidazol-2-yl)-1-phenyl-3-(3-pyridinyl)-2-propen-1-one; 5,6-dimethyl-N-[(5-nitro-2-furyl)methylene]-1H-benzimidazol-1-amine; 11-(5-hydroxy-2-nitrobenzyl)-7,11-diazatricyclo[7.3.1.0˜2,7˜]trideca-2,4-dien-6-one; 6-chloro-2-trichloromethyl)-4H-chromen-4-one; N-(5-bromo-2-biphenylyl)acetamide; diethyl [(1,3-dioxo-2,3-dihydro-1H-inden-5-yl)methylene]malonate; 5-ethylphenazin-5-ium ethyl sulfate; or 3-fluoro-N′-[(5-nitro-2-thienyl)methylene]benzohydrazide.

38. A compound of Formula (I): 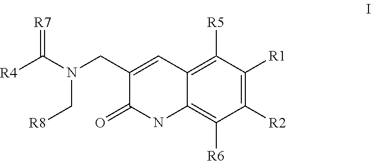
wherein R1 and R2 are independently hydrogen, alkyl, or alkyloxy; or R1 and R2 taken together is methylenedioxy or ethylenedioxy;
R8 is thienyl, furyl or 4-halophenyl;
R4 is 4-alkylphenyl, 2,4-dialkoxyphenyl, 4-alkylphenylamino, 4-alkoxyphenylamino, benzylamino, furylalkylamino or thienyl;
R5 and R6 are independently hydrogen or alkyl;
and R7 is sulfur or oxygen.

39. The compound of claim 38 wherein R1 and R2 are independently hydrogen, ethoxy, methoxy or methyl; or R1 and R2 taken together is ethylenedioxy; and/or R8 is 2-thienyl, 2-furyl or 4-fluorophenyl; and/or R4 is 4-methylphenyl, 4-t-butyl-phenyl, 2,4-dimethoxyphenyl, 4-methylphenylamino, 4-ethoxyphenylamino, benzylamino, furylmethylamino or thienyl.

40. A compound of Formula (II): 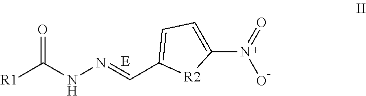
wherein R1 is aryl optionally substituted by one or more halo, alkyl or arylalkyl; and
R2 is oxygen or sulfur.

41. The compound of claim 40 wherein R1 is phenyl optionally substituted with one or more halo or alkyl, such as 4-halophenyl or 3-halophenyl; and/or R1 is 4-(1,1′-biphenyl), naphthyl such as 1-naphthyl, phenylalkyl. Phenylethyl, or naphthylalkyl such as 1-naphthylmethyl.

42. A compound of Formula (III): 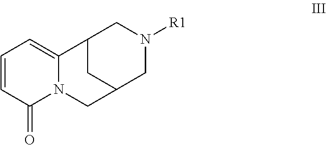
wherein R1 is benzyl optionally substituted with one or more halo, alkoxy, hydroxy, nitro, alkylthio, ethylenedioxy, aryl or benzyloxy groups, or R1 is a 1,3a-dihydropyrenylmethyl.

43. The compound of claim 42 wherein R1 is benzyl substituted with one or more halo, alkoxy or hydroxy groups, such as a halo and an alkoxy group.

44. A compound of Formula (IV): 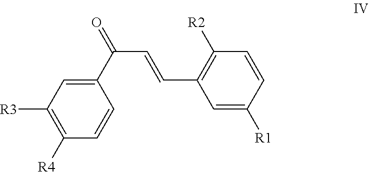
wherein R1 is halo or alkyloxy;
R2 and R3 are independently alkyloxy; and
R4 is hydrogen, alkyl or alkoxy.

45. The compound of claim 44 wherein R1 is bromo or methoxy; and/or R2 is methoxy or ethoxy; and/or R3 is methoxy; and/or R4 is methyl or methoxy.

46. A pharmaceutical composition comprising a compound of any one of claims 38-45.

47. A therapeutic combination comprising an IL12b inhibitor and an inhibitor of cyclic AMP breakdown.

48. The therapeutic combination of claim 47 which is a synergistic combination.


SEQUENCE LISTING (1 sequence)

SEQ ID NO: 1 - DNA, 17 bp, Homo sapiens
  source 1..17: mol_type = genomic DNA
atgttgcaacaagtcag